(19) United States
(12) Patent Application Publication
(10) Pub. No.: US 2011/0305703 A1
(43) Pub. Date: Dec. 15, 2011

(54) ISOLATED VEGF-C AND VEGF-D PEPTIDES AND USES THEREOF

(75) Inventors: Marc G. Achen, Victoria (AU); Steven A. Stacker, Victoria (AU); Natalia Davydova, Victoria (AU)

(73) Assignee: Vegenics Pty Limited, Toorak (AU)

(21) Appl. No.: 13/133,559

(22) PCT Filed: Dec. 8, 2009
(86) PCT No.: PCT/AU2009/001595

§ 371 Date: Aug. 22, 2011

Related U.S. Application Data
(60) Provisional application No. 61/120,807, filed on Dec. 8, 2008.

(51) Int. Cl. A61K 39/395; C07K 7/08; C07K 14/475; C12N 15/12; C07K 16/22; C07K 1/00; A61K 39/00; G01N 33/566; C40B 30/04; A61P 37/04; A61P 35/00; A61P 19/02; A61P 29/00; A61P 17/06; C07K 7/06

(52) U.S. Cl. 424/139.1; 530/330; 530/329; 530/328; 530/327; 530/326; 530/325; 530/324; 536/23.5; 530/387.9; 424/185.1; 436/501; 506/9; 435/7.92; 435/7.24

(57) ABSTRACT

The invention relates to an isolated peptide, comprising residues 147 to 151 of SEQ ID NO: 1, or a modification thereof, and antibodies thereto and uses thereof, wherein the peptide comprises 100 or fewer amino acids.

### FIELD

The invention relates to isolated peptides from VEGF-D and uses thereof.

### BACKGROUND

The most recently discovered mammalian member of the VEGF family, VEGF-D, activates the cell surface receptor tyrosine kinases VEGFR-2 and VEGFR-3, which are expressed on the endothelial cells of blood vessels and lymphatic vessels during embryogenesis, tumor development and other disease states. VEGFR-2, predominantly expressed on blood vessels, is thought to be a critical molecule for signaling angiogenesis, whereas VEGFR-3, expressed on lymphatic vessels, signals for lymphangiogenesis. VEGFR-3 can also be upregulated on growing blood vessels, and participates in angiogenic signaling.

Angiogenesis is a fundamental process required for normal growth and development of tissues, and involves the proliferation of new capillaries from pre-existing blood vessels. Angiogenesis is not only involved in embryonic development and normal tissue growth, repair, and regeneration, but is also involved in the female reproductive cycle, establishment and maintenance of pregnancy, and in repair of wounds and fractures. In addition to angiogenesis which takes place in the normal individual, angiogenic events are involved in a number of pathological processes, notably tumor growth and metastasis, and other conditions in which blood vessel proliferation, especially of the microvascular system, is increased, such as diabetic retinopathy, psoriasis and arthropathies. Recent studies have demonstrated the critical role of angiogenesis in tumor development and the formation of metastatic tumor deposits. The inhibition of tumor angiogenesis has emerged as a promising new therapeutic modality.

The lymphatic vasculature transports fluid and macromolecules from tissues back to the blood circulation. It forms a unidirectional network that collects interstitial fluid to be returned to the venous circulation via collecting vessels, lymph nodes, lymphatic trunks and ducts.

Lymphangiogenesis is the formation of lymphatic vessels, particularly from pre-existing lymphatic vessels. The lymphatic vasculature also links tissue fluids to lymph nodes as an immune surveillance system by which lymphocytes and antigen-presenting dendritic cells travel from peripheral tissues to the lymphoid organs, in which immune responses against pathogens are launched.

VEGF-D promoted tumor angiogenesis and lymphangiogenesis in a mouse model of cancer, facilitating growth of the primary tumor and spread of tumor cells via the lymphatic vessels to lymph nodes. It is expressed in a range of prevalent human cancers and has been reported to be a prognostic indicator of lymphatic involvement and poor patient outcome in some tumor types. Hence, VEGF-D is being explored as a target for novel anti-cancer therapeutics designed to restrict the growth and spread of cancer.

VEGF-D is closely related in structure to the angiogenic and lymphangiogenic protein VEGF-C, which has also been implicated in the growth or spread of cancer. Therefore, drugs targeting VEGF-C are also of interest as anti-cancer therapeutics.

A need exists for an effective, alternative means to prevent, restrict or treat a condition responsive to neutralizing VEGF-D, VEGF-C, or both VEGF-D and VEGF-C, particularly cancer.

### SUMMARY OF THE INVENTION

We have now identified the peptide region within mature human VEGF-D polypeptide to which a neutralizing monoclonal antibody (mAb) specifically binds thus blocking binding of mature human VEGF-D to both VEGFR-2 and VEGFR-3 and inhibiting the associated biological activity of VEGF-D.

According to a first aspect, the invention provides an isolated peptide, comprising residues 147 to 151 of SEQ ID NO: 1, wherein the peptide comprises 100 or fewer amino acids.

According to a second aspect, the invention provides an isolated peptide, comprising residues 147 to 151 of SEQ ID NO: 1 and a modification that is not N147del, E148del, E149del, S150del, L151del, N147Q, E148A, E148D, E149D, S150C, S150T, S150G or L151V, wherein the peptide comprises 100 or fewer amino acids.

In one embodiment according to the second aspect, one or more of residues 147, 148, 149, 150 and 151 of SEQ ID NO: 1 is/are modified. Such embodiments may provide a peptide that has greater amino acid sequence identity with VEGF-C and which may be bound by a VEGF-D neutralizing antibody, or may allow generation of an antibody that is cross-reactive with, specifically binds, and neutralizes both VEGF-D and VEGF-C.

Identification of the VEGF-D peptide to which a VEGF-D neutralizing antibody specifically binds enables prediction and generation of the homologous peptide in VEGF-C that is important for antibody binding and/or receptor binding. Residues 147 to 151 of human VEGF-D (SEQ ID NO: 1) are homologous to residues 167 to 171 of human VEGF-C (SEQ ID NO: 2). Although polypeptide fragments from VEGF-C have been described previously, the amino acid residues important as far as an antibody neutralizing the activity of VEGF-C have not identified.

According to a third aspect, the invention provides an isolated peptide, comprising residues 167 to 171 of SEQ ID NO: 2, wherein the peptide comprises 100 or fewer amino acids.

According to a fourth aspect, the invention provides an isolated peptide, comprising residues 167 to 171 of SEQ ID NO: 2 and a modification that is not N167del, E169del, or L171del (corresponding with residues 147, 149 and 151, respectively, of SEQ ID NO: 1), wherein the peptide comprises 100 or fewer amino acids.

In one embodiment according to the fourth aspect, one or more of residues 167, 168, 169, 170 and 171 of SEQ ID NO: 2 is/are modified. Such embodiments may provide a peptide that has greater amino acid sequence identity with VEGF-D and which may be bound by a VEGF-C neutralizing antibody, or may allow generation of an antibody that is cross-reactive with, specifically binds, and neutralizes both VEGF-C and VEGF-D.

In a preferred embodiment of the first, second, third and fourth aspects, the isolated peptides comprise one or more amino acid residues in addition to the wild-type or altered regions corresponding to positions 147-151 of VEGF-D, in the case of the first and second aspects, or positions 167-171 of VEGF-C, in the case of the third and fourth aspects.

As described in greater detail below, preferred peptides of the invention have useful immunological properties. For example, antibodies that bind to the peptide (e.g., when the peptide is used as an antigen in the generation of antibodies, or used for panning a library of antibodies or antibody segments) also bind the respective ligand, VEGF-D and/or VEGF-C. Such antibodies that bind the peptide and bind the respective ligand may be useful for inhibiting or neutralizing one or more biological activities of the respective ligand, such as VEGF-D binding to one or more of its receptors, VEGFR-2 and VEGFR-3.

Preferably, the peptide is immunogenic in vivo in mammals, including humans.

According to a fifth aspect, the invention provides a nucleic acid molecule encoding a peptide according to any one of the first, second, third and fourth aspects of the invention.

Thus, the invention also relates to nucleic acid molecules encoding a peptide region derived from mature human VEGF-D or VEGF-C polypeptide to which VEGF-D or VEGF-C neutralizing antibody binds.

Related embodiments of the invention include nucleic acid constructs wherein the nucleic acid that encodes the peptide is operatively attached to one or more expression control sequences; vectors (including expression vectors and gene therapy vectors) that comprise such nucleic acid molecules and constructs; and isolated cells transformed or transfected or otherwise containing the nucleic acids, constructs, or vectors.

In a sixth aspect, the invention relates to an antibody that is raised against a peptide as defined in any one of the first, second, third and fourth aspects of the invention, wherein the antibody specifically binds VEGF-D or VEGF-C.

In an embodiment of the sixth aspect of the invention, the antibody is capable of specifically binding VEGF-D and VEGF-C.

According to a seventh aspect, the invention relates to use of an isolated peptide as defined in any one of the first, second, third and fourth aspects of the invention for the generation of an antibody that specifically binds VEGF-D or VEGF-C.

According to an eighth aspect, the invention relates to use of an isolated peptide as defined in the second or fourth aspects of the invention for the generation of an antibody that specifically binds one or more of the peptides according to any one of the first, second, third and fourth aspects.

The invention also encompasses use of an antibody generated according to the invention, whereby such an antibody binds specifically to a peptide or peptide variant derived from VEGF-D or VEGF-C.

It is contemplated that identification of the VEGF-D and VEGF-C peptides responsible for specifically binding VEGF-D and VEGF-C neutralizing antibodies, respectively, will allow generation of new anti-VEGF-D and anti-VEGF-C neutralizing antibodies. Furthermore, by introducing one or more amino acid modifications into the VEGF-D or VEGF-C peptide, the variant peptide may enable generation of an antibody that specifically binds to and neutralizes native VEGF-D and/or VEGF-C polypeptide. In one embodiment, the modification comprises substitution of a homologous residue of VEGF-C into the VEGF-D peptide. In another embodiment, the modification comprises substitution of a homologous residue of VEGF-D into the VEGF-C peptide.

Alternatively, phage display technology may allow isolation of an antibody that binds to and neutralizes native VEGF-D and/or VEGF-C polypeptide without modification of an amino acid.

The invention also includes methods of generating an antibody. For example, one embodiment comprises the method of immunizing an animal one or more times with a composition that includes a peptide of the invention (and optionally includes additional components such as a pharmaceutically acceptable adjuvant and/or carrier); and isolating from the animal an antibody or an antibody-producing cell, wherein the antibody binds the peptide and binds VEGF-D and/or VEGF-C. A further embodiment comprises the method of contacting one or more compounds from a library, such as a phage display library, with a peptide of the invention; and isolating a member from the library that binds the peptide. Optionally, such methods further include one or more steps of assaying the antibody or library member that binds the peptide for VEGF-D or VEGF-C binding or VEGF-D/VEGF-C neutralization activities, and selecting an antibody that has said binding and/or said neutralizing activity.

According to a ninth aspect, the invention relates to an immunogenic composition or a vaccine for eliciting an immune response, comprising (a) an isolated peptide according to any one of the first, second, third and fourth aspects or (b) a nucleic acid molecule encoding a peptide according to (a). The composition or vaccine optionally includes additional components, such as an adjuvant, as described below in detail.

Peptides comprising the VEGF-D or VEGF-C antigen responsible for VEGF-D or VEGF-C neutralizing antibody specific binding, i.e. comprising residues 147 to 151 of SEQ ID NO: 1 or residues 167 to 171 of SEQ ID NO: 2, or a variant thereof, may be used to generate in vivo an immune response to VEGF-D or VEGF-C. Preferably, the immune response comprises neutralizing antibodies that block interactions of VEGF-D or VEGF-C with their receptors. Alternatively, a vaccine may include a peptide variant comprising an amino acid modification according to the invention such that the in vivo immune response comprising neutralizing antibodies is sufficient to reduce interactions between VEGF-D and/or VEGF-C and their receptors.

According to a tenth aspect, the invention relates to use of:
(a) an isolated peptide according to any one of the first, second, third and fourth aspects;
(b) a nucleic acid molecule encoding a peptide according to (a); or
(c) an antibody that is raised against a peptide according to (a) and specifically binds VEGF-D or VEGF-C,
   in the manufacture of a medicament for treating a condition responsive to neutralizing VEGF-D, VEGF-C, or both VEGF-D and VEGF-C.

According to an eleventh aspect, the invention relates to a method for treating a condition responsive to neutralizing VEGF-D, VEGF-C, or both VEGF-D and VEGF-C, comprising the step of administering to a subject in need thereof a therapeutically effective amount of:
(a) an isolated peptide according to any one of the first, second, third and fourth aspects;
(b) a nucleic acid molecule encoding a peptide according to (a); or
(c) an antibody that is raised against a peptide according to (a) and specifically binds VEGF-D or VEGF-C.

In an embodiment of the tenth and eleventh aspects, the condition comprises dysregulated angiogenesis, dysregulated lymphangiogenesis, cancer, rheumatoid arthritis, psoriasis, lymphangiolieomyomatosis or other inflammatory conditions.

Thus, peptides of the invention, nucleic acid molecules encoding peptides of the invention, or antibodies of the invention can be used in the manufacture of a medicament, or may be administered in one step of a method of treatment, in relation to a condition responsive to neutralizing VEGF-D, VEGF-C, or both VEGF-D and VEGF-C.

In a twelfth aspect, the invention provides use of:
(a) an isolated peptide according to the first or third aspect; or
(b) an antibody that is raised against a peptide according to (a) and specifically binds VEGF-D or VEGF-C,
   for determining an amino acid residue of VEGF-D or VEGF-C required for specific binding of VEGF-D or VEGF-C to a receptor.

In a thirteenth aspect, the invention provides use of:
(a) an isolated peptide according to the first or third aspect; or
(b) an antibody that is raised against a peptide according to (a) and specifically binds VEGF_D or VEGF-C,
   for determining an amino acid residue of a receptor required for specific binding of a ligand to the receptor.

In one embodiment of the twelfth and thirteenth aspects, the receptor is VEGFR-2 or VEGFR-3. In another embodiment, the receptor is NRP-1, NRP-2, or an integrin, particularly integrin α9β1.

Knowledge of the peptides of VEGF-D or VEGF-C to which an antibody binds specifically may also allow determination of the critical amino acid residues required for specific binding of VEGF-D or VEGF-C to a receptor. In turn, such knowledge may enable determination of a critical amino acid residue of a receptor that is required for specific binding of a ligand, including an agent that partially or fully blocks, neutralizes, reduces or antagonizes a biological activity of VEGF-D, VEGF-C, or both VEGF-D and VEGF-C.

The original claims appended hereto are hereby incorporated by reference into the summary of the invention. Inventions described as uses should be considered to also constitute a description of methods or processes of using, and vice versa, in view of different jurisdictions preferences for characterizing inventions differently.

The foregoing summary is not intended to define every aspect of the invention, and the heading “Summary of the Invention” is not intended to be limiting in any way; additional aspects of the invention and further details of the invention are described in other sections, such as the Drawings or Detailed Description. The entire document is intended to be related as a unified disclosure, and it should be understood that all combinations of features described herein are contemplated, even if the combination of features are not found together in the same sentence, or paragraph, or section of this document. To provide an example, where protein therapy is described, embodiments involving polynucleotide (nucleic acid) therapy (using polynucleotides/nucleic acids that encode the protein) are always specifically contemplated, and the reverse also is true.

In addition to the foregoing, the invention includes, as an additional aspect, all embodiments of the invention narrower in scope in any way than the embodiments specifically mentioned above. Embodiments summarized in the Summary section are frequently further described, with preferred variations, in the Detailed Description, and these variations are part of the invention incorporated into the Summary by reference. With respect to aspects of the invention described as a genus, all individual species are individually considered separate aspects of the invention. With respect to aspects of the invention that are described with reference to exemplary numerical values, it should be understood that such values are intended to describe ranges or sub-ranges that include the recited values. With respect to aspects described with numerical ranges, it should be understood that all subranges are contemplated. With respect to peptide lengths summarized with ranges or limits, all integer lengths within the ranges or limits are specifically contemplated as species of the invention.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWINGS

FIG. 1 provides an amino acid sequence of human VEGF-D (SEQ ID NO: 1). The VHD of human VEGF-D is underlined. Residues 89 to 93 of VEGF-D represent the sequence identified as the amino-terminus of the predominant form of mature VEGF-D.

FIG. 2 provides an amino acid sequence of human VEGF-C (SEQ ID NO: 2). The VHD of human VEGF-C is underlined.

FIG. 3 illustrates a comparison of human (h) VEGF-D with other members of the VEGF family. Alignment of the deduced amino acid sequences of hVEGF-D, mouse (m) VEGF-D, hVEGF-C, hVEGF165, hVEGF-B167, and hPlGF is shown. Residues that match the sequence of hVEGF-D are boxed. Asterisks above the hVEGF-D sequence denote the eight cysteine residues that are conserved in all VEGF family members. Arrows denote positions of proteolytic cleavage that give rise to mature hVEGF-C.

FIG. 4A provides an amino acid sequence for the VHD of human VEGF-D (SEQ ID NO: 3). The N-terminal is residue 89 (F) and the C-terminal is residue 205 (R), according to the numbering of FIGS. 1 and 3. FIG. 4B lists the amino acid sequences of individual peptides in the first library used for mapping the VEGF-D neutralizing antibody-binding site. “GSG” is a C-terminal linker between the peptide and biotin. “SGSG” is an N-terminal linker between biotin and the peptide. FLAG sequence is in bold typeface. Residue numbering is relative to SEQ ID NO: 1.

FIG. 5A depicts the result of peptide mapping of the VEGF-D neutralizing antibody-binding site by ELISA using the first peptide library of FIG. 4. FIG. 5B depicts the ELISA result of M2 anti-FLAG mAb control for non-specific binding. The peptide identifier is presented on the x-axes; the assay response, absorbance at 405 nm, is presented on the y-axes.

FIG. 6 lists the amino acid sequences of individual peptides in the second library used for mapping the VEGF-D neutralizing antibody-binding site. “SGSG” is an N-terminal linker between biotin and the peptide. Residue numbering is relative to SEQ ID NO: 1. Peptides 22 to 28 (SEQ ID NOs: 61 to 67) comprise one or more mutations in which cysteine has been replaced by serine. Peptide 30 (SEQ ID NO: 69) from mouse VEGF-D is homologous to Peptide 31 (SEQ ID NO: 70) from Human VEGF-D. Peptide 31 (SEQ ID NO: 70) is identical to Peptide 17 (SEQ ID NO: 20) from the first library (see FIG. 4B). Peptides 32 to 35 (SEQ ID NOs: 71 to 74) comprise peptides from human VEGF-family members that are homologous to Peptide 31 (SEQ ID NO: 70).

FIG. 7A illustrates the result of peptide mapping of VEGF-D neutralizing antibody-binding site by ELISA using the second peptide library of FIG. 6. FIG. 7B depicts the ELISA result of M2 anti-FLAG mAb control for non-specific binding. The peptide identifier is presented on the x-axes; the assay response, absorbance at 405 nm, is presented on the y-axes.

FIG. 8 lists the amino acid sequences of individual peptides in the third library used for mapping the VEGF-D neutralizing antibody-binding site. “SGSG” is an N-terminal linker between biotin and the peptide. “SGS” is a C-terminal linker between the peptide and biotin. “Bio” refers to biotin. Residue numbering is relative to SEQ ID NO: 1. Peptides 60, 61, 62, 63, 64, 65 and 66 (SEQ ID NOs: 134 to 140) are identical to peptides 17, 18, 24, 25, 26, 32 and 33 (SEQ ID NOs: 20, 21, 27, 28, 29, 35 and 36, respectively) of the first peptide library (FIG. 4B), respectively. Peptide 5 (SEQ ID NO: 79) of the third library is identical to peptide 14 (SEQ ID NO: 53) of the second library (FIG. 6). Peptide 6 (SEQ ID NO: 80) of the third library is similar to peptide 14 (SEQ ID NO: 53) of the second library; the biotin residue of peptide 6 is at the C-terminal, rather than the N-terminal, as for peptide 5. Furthermore, the third library comprises peptides related to peptide 14 (SEQ ID NO: 53) of the second library. Such related peptides comprise: truncated variants with N-terminal or C-terminal biotin residues (peptides 11 to 16, SEQ ID NOs: 85 to 90); peptides with amino acid substitutions or deletions (peptides 1 to 4, 7 to 10, 17 to 48, 58 and 59, SEQ ID NOs: 75 to 78, 81 to 84, 91 to 122, 132 and 133, respectively); and peptides from homologous regions of other VEGF family members with amino acid substitutions to make these regions more like VEGF-D (peptides 49 to 57, SEQ ID NO: 123 to 131).

FIG. 9A illustrates the result of peptide mapping of VEGF-D neutralizing antibody-binding site by ELISA using the third peptide library of FIG. 8. FIG. 9B depicts the ELISA result of M2 anti-FLAG mAb control for non-specific binding. The peptide identifier is presented on the x-axes; the assay response, absorbance at 405 nm, is presented on the y-axes.

FIG. 10 provides an amino acid sequence of five amino acid residues (SEQ ID NO: 144) derived from human VEGF-D according to FIG. 1 (residues 147 to 151 of SEQ ID NO: 1) that may be important for specific binding of a VEGF-D neutralizing antibody.

FIG. 11 provides an amino acid sequence of VEGF-DΔNΔC (SEQ ID NO: 145). VEGF-DΔNΔC comprises residues 93 to 201 of FIG. 1 (human VEGF-D; SEQ ID NO: 1) and the FLAG octapeptide at the N-terminus.

FIG. 12 lists the nucleotide sequences of mutagenesis primers used to generate VEGF-DΔNΔC variants derived from VEGF-DΔNΔC of FIG. 11.

FIG. 13 depicts Western blot analyses of conditioned media containing VEGF-DΔNΔC variants of FIG. 12. FIG. 13A depicts detection with M2 antibody that binds the FLAG epitope. FIG. 13B depicts detection with a neutralizing antibody that binds VEGF-D. Each lane was loaded with 5 from a total of 30 ml conditioned media. M—Molecular weight markers (kDa).

FIG. 14 illustrates binding of a VEGF-D neutralizing antibody to the VEGF-DΔNΔC variants of FIG. 12 in sandwich ELISA. M2 antibody, targeting the FLAG epitope, was used to capture the VEGF-DΔNΔC variants, and either control VEGF-D antibody MAB286 (FIG. 14A) or a VEGF-D neutralizing antibody (FIG. 14B) was used for detection. X-axes define the VEGF-DΔNΔC variants. Y-axes represent level of binding of the detection antibody to captured VEGF-DΔNΔC variants compared with binding to VEGF-DΔNΔC (expressed as a percentage). The data is mean±standard deviation (n=3).

FIG. 15 depicts Western blot analyses of conditioned media containing human VEGF-DΔNΔC variants of FIG. 12 with “mouse” mutations. FIG. 15A depicts detection with M2 antibody that binds the FLAG epitope. FIG. 15B depicts detection with a neutralizing antibody that binds VEGF-D. Each lane was loaded with 5 μl from a total of 30 ml conditioned media. M—Molecular weight markers (kDa).

FIG. 16 provides an amino acid sequence of a 15-mer peptide comprising five amino acid residues derived from human VEGF-D (SEQ ID NO: 144) according to FIG. 1 (residues 147 to 151 of SEQ ID NO: 1) used for generating rabbit anti-peptide antibodies (“rabbit NEESL” antisera).

FIG. 17 illustrates that affinity-purified rabbit NEESL generated against the peptide of FIG. 16 binds to VEGF-DΔNΔC in ELISA. The X-axis defines the concentration of rabbit NEESL. The Y-axis represents detection of the secondary antibody via absorbance at 405 nm. Binding of secondary antibody to VEGF-DΔNΔC in the absence of affinity-purified rabbit NEESL is shown as the negative control.

FIG. 18 shows that rabbit NEESL generated against the peptide of FIG. 16 blocks binding and cross-linking by VEGF-D to VEGFR-2 or VEGFR-3 extracellular domains. The X-axis defines the VEGFR-2 or VEGFR-3 extracellular domain. The Y-axis represents ³H-thymidine incorporation relative to the positive control. The data is mean±standard error (n=2).

FIG. 19 provides an amino acid sequence of five amino acid residues (SEQ ID NO: 161) derived from human VEGF-C according to FIG. 2 (residues 167 to 171 of SEQ ID NO: 2) that may be important for specific binding of a VEGF-C neutralizing antibody.

### DETAILED DESCRIPTION

Various strategies may be used to inhibit the function of polypeptides such as VEGF-D or VEGF-C. These include targeting the cognate receptor (VEGFR-2 or VEGFR-3) to inhibit ligand binding, using gene therapy techniques that deliver antisense oligonucleotides to inhibit expression of the ligand or receptor, use of soluble VEGFR-2 or VEGFR-3 to inhibit ligand binding to the receptor, development of receptor tyrosine kinase inhibitors to inhibit receptor-mediated signal transduction, and monoclonal antibodies (mAb) directed against VEGF-D or VEGF-C to inhibit ligand binding to the receptor. A recombinant humanized version of a murine anti-human mAb (rhuMab VEGF, Bevacizumab™) targeting VEGF-A, another member of the VEGF family, has been produced and used in subjects with metastatic cancer.

Use of immunogens or vaccines to prevent or treat cancer is an attractive approach because of the expected minimal side effects of vaccine therapy. Strategies for immunization have included whole cell vaccines, protein and DNA vaccines, as well as peptide vaccines; each type of anti-tumor vaccine has its advantages and limitations. Peptides are an attractive anti-cancer vaccine in that they are considered safe (free of pathogens and oncogenic potential), stable, easily constructed, and are cost-effective. Importantly, peptide vaccines lead to sustained immune responses and memory, unlike the response generated from passive immunization.

VEGF-C and VEGF-D are two of six members of a family of angiogenic regulators. Other members are VEGF-A (also known as VEGF), VEGF-B, VEGF-E and placental growth factor (PlGF). VEGF-C and VEGF-D are synthesized as precursor proteins containing N-terminal and C-terminal propeptides in addition to the central VEGF homology domain (VHD) which contains the binding sites for VEGFR-2 and VEGFR-3. The propeptides are proteolytically cleaved from the VHD during biosynthesis by proprotein convertases to generate a mature form. In humans, the mature proteolytically processed forms of VEGF-C and VEGF-D bind to VEGFR-2 and VEGFR-3. In mice, VEGF-D binding is restricted to VEGFR-3.

VEGF-C and VEGF-D exist as homodimers, and it has been suggested that they may exist as VEGF-C-VEGF-D heterodimers.

VEGF-C and VEGF-D specifically bind to and activate VEGFR-3, such that VEGFR-3-mediated signaling appears to be central to the regulation of lymphangiogenesis.

“Lymphangiogenesis” refers to formation of lymphatic vessels, particularly from pre-existing lymphatic vessels.

During embryogenesis, lymphatic endothelial cell sprouting, proliferation and survival is promoted by VEGF-C. Lymphatic vessels fail to develop in mice in which VEGF-C is absent (Vegfc knockout mice), and such mice develop severe edema. Indeed, absence of VEGF-C is embryonic lethal. Lymphatic vessel hypoplasia and lymphedema is exhibited in the skin of mice hemizygous for Vegfc (i.e. mice possessing one functional allele).

Similar to VEGF-C, VEGF-D also partly regulates lymphangiogenesis. However, lymphangiogenesis during embryonic development is not dependent upon VEGF-D, as demonstrated by Vegfd knockout mice, which is in contrast to VEGF-C. The lymphatic system in Vegfd knockout mice is relatively normal and Vegfd knockout mice are viable and fertile. However, the absolute abundance of lymphatic vessels in the lung, for example, is reduced by approximately 30% compared to wild-type mice.

In addition to lymphatic vessels, VEGFR-3 is also expressed on blood vessel endothelial cells during development, thereby accounting for the severe vasculogenic and angiogenic defects observed during early embryogenesis in models comprising inactive VEGFR-3 signaling. The lymphatic system possesses almost exclusive expression of VEGFR-3 in adulthood, because VEGFR-3 expression in blood vessels declines following birth and during adolescence. Thus, only lymphangiogenesis is inhibited in adults by inhibition of the VEGF-C/VEGF-D-VEGFR-3 signaling axis.

VEGF-C and VEGF-D can bind specifically to neuropilin-(NRP-2) which is expressed by lymphatic vessels. The binding affinity of VEGF-C or VEGF-D to VEGFR-3 is thought to be increased by NRP-2 acting as a co-receptor for VEGFR-3 in lymphangiogenesis. NRP-2 is required for lymphangiogenesis. Proliferation of lymphatic vessel endothelial cells was reduced and lymphatic vessels and capillaries failed to develop in Nrp2 knockout mice in which NRP-2 is absent. Similarly, NRP-1 is capable of binding VEGF-C and VEGF-D.

VEGF-C and VEGF-D may act as ligands for integrins. Specifically, VEGF-C and VEGF-D have been shown to act as ligands for integrin α9β1. Cell adherence and cell migration were promoted by each of VEGF-C and VEGF-D in cells expressing integrin α9β1. The effect could be blocked by an anti-integrin α9β1 antibody or siRNA directed to integrin α9β1. Consequently, it is thought that binding of VEGF-C or VEGF-D to integrins, particularly integrin α9β1, also performs a role in lymphangiogenesis.

VEGF-D and VEGF-C link lymphangiogenesis to angiogenesis and can stimulate both processes. Lymphangiogenesis is thought to be driven primarily through activation of VEGFR-3. Angiogenesis is thought to be driven primarily through activation of VEGFR-2. Both VEGF-D and VEGF-C promote in vitro endothelial cell proliferation and migration. VEGF-C stimulates vascular permeability, which is thought to occur via VEGFR-2. Anti-VEGFR-2 antibodies were able to ablate the vascular endothelial cell migratory effect induced by VEGF-D. VEGF-D and VEGF-C induce angiogenesis, at least in tumor models, which could be blocked by anti-VEGFR-2 antibodies.

“Angiogenesis” refers to formation of blood vessels, particularly the proliferation of new capillaries from pre-existing blood vessels.

Importantly, the lymphangiogenesis induced by VEGF-D and VEGF-C promotes metastatic spread of tumor cells to the lymphatic vessels and lymph nodes, and the angiogenesis induced by VEGF-D and VEGF-C in tumors can promote solid tumor growth and metastatic spread. Furthermore, clinicopathological data indicates a role for these growth factors in a range of prevalent human cancers. For example, VEGF-D expression was reported to be an independent prognostic factor for both overall and disease-free survival in colorectal cancer and levels of VEGF-C mRNA in lung cancer are associated with lymph node metastasis and in breast cancer correlate with lymphatic vessel invasion and shorter disease-free survival.

Peptides and Variants

The peptides of this invention relate to the representative peptides as described herein, and to functional variants of these peptides.

“Peptide”, “polypeptide”, and “protein” can be used interchangeably and encompass functional variants of such peptides.

“Functional variant”, “functional peptide variant”, “peptide variant”, or “variant” as used herein can be used interchangeably and includes either natural peptide variants or artificially modified peptide variants that immunologically mimic the VEGF-D or VEGF-C peptide described above. Such artificially modified variants can be made by synthetic chemistry of recombinant DNA mutagenesis techniques that are well known to persons skilled in the art.

In a preferred embodiment of the first and second aspects, the isolated peptides comprise one or more amino acid residues in addition to the wild-type or modified regions corresponding to positions 147-151 of VEGF-D. In a preferred embodiment of the third and fourth aspects, the isolated peptides comprise one or more amino acid residues in addition to the wild-type or modified regions corresponding to positions 167-171 of VEGF-C.

Preferably, the peptide has an amino acid sequence that is at least 60%, at least 65%, at least 70%, at least 75%, at least 80%, at least 85%, at least 90%, at least 95%, at least 96%, at least 97%, at least 98%, at least 99% or is 100% identical over its length to a segment (preferably a continuous segment) of a wild-type VEGF-D sequence, preferably the human VEGF-D sequence of SEQ ID NO: 1, or VEGF-C sequence, preferably the human VEGF-C sequence of SEQ ID NO: 2.

Peptides of the invention are suitably at least 5, preferably at least 6 and more preferably at least 7 amino acid residues in length. Peptides may be 8, 9, 10, 11, 12, 13, 14, 15, 16, 17, 18, 19, 20, 21, 22, 23, 24, 25, 26, 27, 28, 29 and 30 amino acids in length, or longer. Peptides of the invention are suitably no longer than 100, preferably no longer than 90 and more preferably no longer than 80 amino acid residues. The peptides of the invention are preferably no longer than 70, 60, 50, 40, 35 or 30 amino acid residues. The peptides may be less than 25, 20 or 15 amino acids in length. Examples of suitable peptides are those consisting of 6 to 25, 7 to 20 or 8 to 15 amino acid residues.

As used herein, “modified” or “modification” includes substitution, replacement, addition, insertion, omission and/or deletion of an amino acid residue.

In some variations, the peptide is comprised in a heterologous fusion construct. In some variations, the modifications to the peptide sequence relative to the reference VEGF-D or VEGF-C sequence consist of conservative substitutions. In some variations, the modifications to the peptide relative to the reference VEGF-D or VEGF-C sequence consist of substituted residues selected from an aligned VEGF-C or VEGF-D sequence, respectively. An exemplary alignment is provided in the Drawings.

A functional variant of the peptide of the invention may have an amino acid sequence identity of at least 60% with SEQ ID NO: 144, which consists of amino acid residues 147 to 151 of SEQ ID NO: 1. Alternatively, a functional variant may have an amino acid sequence identity of at least 60% with SEQ ID NO: 157, which consists of amino acid residues 167 to 171 of SEQ ID NO: 2. Alternatively, the functional variant may have at least about 80% or 100% amino acid sequence identity with SEQ ID NO: 144 or 157. In any case, the functional variant will still be capable of immunologically mimicking the corresponding antigenic determinant site of the human VEGF-D or VEGF-C protein.

VEGF-D and VEGF-C peptides also encompass peptides that are antigenic and variants of the peptides provided as SEQ ID NOs: 20, 70 or 134 (excluding biotin and the linker sequence). Such variants have a modified sequence in which an amino acid in the corresponding reference sequence is substituted, or in which an amino acid is deleted from or added to the reference sequence.

A functional variant of the peptide of the invention may have an amino acid sequence identity of at least about 60% with one of SEQ ID NOs: 20, 70 or 134 (excluding biotin and the linker sequence). Alternatively, the functional variant may have at least about 65%, 70%, 75%, 80%, 85%, 90%, or 95%, or at least about 99% amino acid sequence identity with one of SEQ ID NOs: 20, 70 or 134 (excluding biotin and the linker sequence). In any case, the functional variant will still be capable of immunologically mimicking the corresponding antigenic determinant site of the human VEGF-D or VEGF-C protein.

The preferred number of modifications in a peptide of the invention to produce a peptide variant is 0, 1, or 2. Peptides with a larger number of modification, for example 3, 4, 5, 6, 7, 8, 9, or 10, or even 15, 20, 25, 30, 35, 40, 45, or 50 are also contemplated by the invention.

For this invention, to “immunologically mimic” the corresponding antigenic determinant site of the VEGF-D or VEGF-C protein is (1) defined as a (variant) peptide being capable of inducing antibodies that specifically recognize one of the wild-type epitope sequences described above in the context of the whole VEGF-D or VEGF-C protein and/or (2) defined as a (variant) peptide being capable of being recognized by the same specific antibody that recognizes one of the VEGF-D or VEGF-C epitopes described above in the context of the whole VEGF-D or VEGF-C protein. In the first definition, the variant peptide should be capable of inducing such antibodies either by itself, or in conjunction with a carrier molecule. In the second definition, the variant peptide should be capable of being recognized either by itself, or in conjunction with a carrier molecule.

A functional peptide variant can be obtained by substitution, replacement, addition, insertion, omission and/or deletion of an amino acid of these amino acid sequences, and/or a functional nucleic acid sequence for producing said amino acid sequences or functional peptide variants. Preferred modifications are amino acid residue substitutions or replacements. In particular, preferred peptide variants have one or more conservative substitutions without losing their property as a functional variant. The peptides or their functional variants can also be linked with other peptides or polypeptides or with further chemical groups such as glycosyl groups, lipids, phosphates, acetyl groups or the like, provided they do not strongly adversely influence their effect.

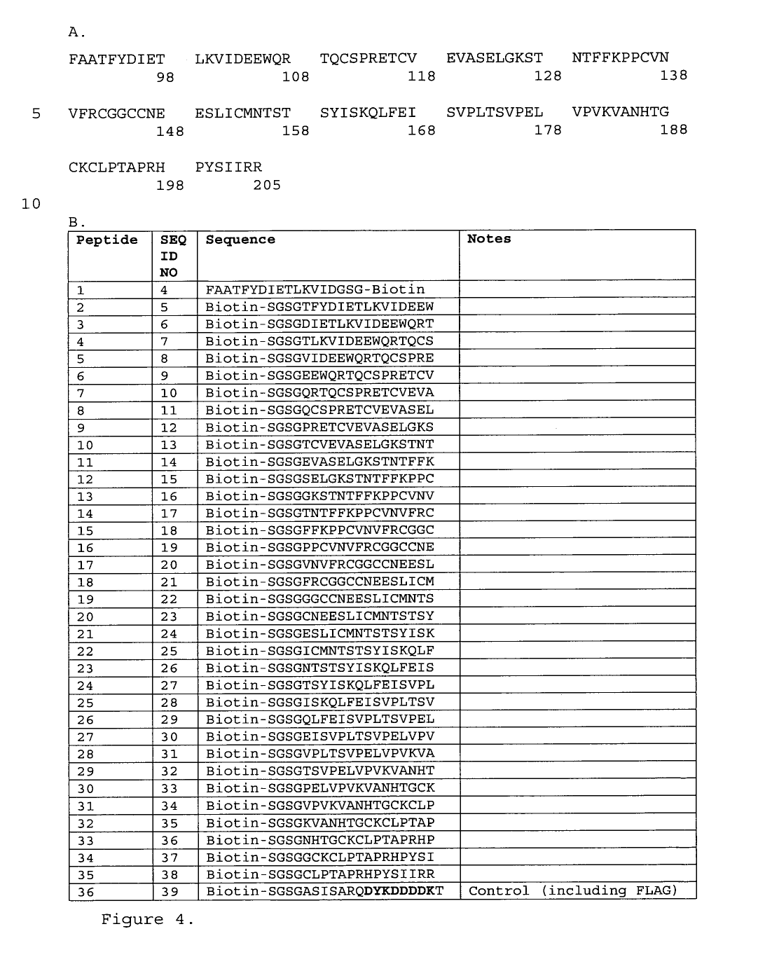

The peptide of the invention may also comprise a non-specific linker that can be adjoined to the peptide. Such a linker is not involved in peptide activity. Rather the linker may serve as a spacer between the peptide sequence and a functional moiety. One example would be a linker used between the peptide and biotin, where biotin is used for immobilization of the peptide. Other uses for a linker include attachment of a moiety to aid purification or detection. Alternatively, and importantly, a linker may allow attachment of a moiety to the peptide that enables specific delivery of the peptide to a particular target, such as a cell or tissue, spatially or temporally. When used as a vaccine, the peptide of the invention may be coupled to a linker that serves as a spacer between the peptide and the immunogenic carrier, or permits improved coupling between the peptide and the immunogenic carrier. An example of a peptide linker (SGSG) is provided in FIGS. 4, 6 and 8.

Thus, the specifically stated peptide sequences can thus vary, provided individual substitution, addition and/or omission of an amino acid does not strongly impair the function of the peptide, i.e. its ability to bind to a VEGF-D neutralizing antibody, or an antibody according to the invention.

In addition to naturally occurring amino acids, non-naturally occurring amino acids, or modified amino acids, are also contemplated and within the scope of the invention. In fact, as used herein, “amino acid” refers to naturally occurring amino acids, non-naturally occurring amino acids, and amino acid analogs, and to the D or L stereoisomers of each.

Natural amino acids include alanine (A), arginine (R), asparagine (N), aspartic acid (D), cysteine (C), glutamine (O), glutamic acid (E), glycine (G), histidine (H), isoleucine (I), leucine (L), lysine (K), methionine (M), phenylalanine (F), proline (P), serine (S), threonine (T), tryptophan (W), tyrosine (Y), valine (V), hydroxyproline (O and/or Hyp), isodityrosine (IDT), and di-isodityrosine (di-IDT). Hydroxyproline, isodityrosine, and di-isodityrosine are formed post-translationally. Use of natural amino acids, in particular the 20 genetically encoded amino acids, is preferred.

The substitutions may be conservative amino acid substitutions, in which the substituted amino acid has similar structural or chemical properties with the corresponding amino acid in the reference sequence. Alternatively, the substitutions may be non-conservative amino acid substitutions.

By way of example, conservative amino acid substitutions involve substitution of one aliphatic or hydrophobic amino acids, e.g., alanine, valine, leucine and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine and histidine, with another; and replacement of one small amino acid, e.g., alanine, serine, threonine, methionine, and glycine, with another.

In one embodiment according to the second aspect of the invention, a substitution in the peptide of the invention may comprise S150A.

In one embodiment according to the fourth aspect of the invention, a substitution in the peptide of the invention may comprise S170A.

Peptide variants may be obtained in which the peptide has been chemically modified at the level of amino acid side chains, of amino acid chirality, and/or of the peptide backbone. These alterations are intended to provide peptides having similar or improved therapeutic, diagnostic and/or pharmacokinetic properties.

For example, when the peptide is susceptible to cleavage by peptidases following injection into a subject, replacement of a particularly sensitive peptide bond with a non-cleavable peptide bond can provide a peptide more stable and thus more useful as a therapeutic agent. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life will be known to the person skilled in the art.

An “agent” or “therapeutic agent” of the invention as used herein refers to a peptide, a nucleic acid molecule, an antibody, or a vaccine according to the invention.

Functional variants of the VEGF-D or VEGF-C peptide of the invention generally may be identified by modifying the sequence of the peptide and then assaying the resulting peptide for the ability to stimulate an immune response, e.g., production of antibodies, or bind to an isolated antibody such as VEGF-D neutralizing antibody.

Preparation of Peptides and Variants

Peptides of the present invention can be prepared in any suitable manner. Such peptides include recombinantly produced peptides, synthetically produced peptides, or peptides produced by a combination of these methods. Means for preparing such peptides are well understood in the art.

The VEGF-D or VEGF-C peptide, or variant thereof, may be synthesized using commercially available peptide synthesizers. The VEGF-D or VEGF-C peptide, or variant thereof, may also be produced using cell-free translation systems and RNA molecules derived from DNA constructs that encode the peptide. Alternatively, the VEGF-D or VEGF-C peptide, or variant thereof, is made by transfecting host cells with expression vectors that comprise a DNA sequence that encodes the respective epitope or chimeric peptide and then inducing expression of the polypeptide in the host cells.

For recombinant production, a recombinant construct comprising a sequence which encodes the peptide, or a variant thereof, is introduced into host cells by conventional methods such as calcium phosphate transfection, DEAE-dextran mediated transfection, transvection, microinjection, cationic lipid-mediated transfection, electroporation, transduction, scrape lading, ballistic introduction or infection.

The VEGF-D or VEGF-C peptide, or variant thereof, may be expressed in suitable host cells, such as for example, mammalian cells, yeast, bacteria, insect cells or other cells under the control of appropriate promoters using conventional techniques. Suitable hosts include, but are not limited to, E. coli, P. pastoris, COS cells, and 293 HEK cells. Following transformation of the suitable host strain and growth of the host strain to an appropriate cell density, the cells are harvested by centrifugation, disrupted by physical or chemical means, and the resulting crude extract retained for further purification of the peptide, or variant thereof.

Conventional procedures for isolating recombinant proteins from transformed host cells, such as isolation by initial extraction from cell pellets or from cell culture medium, followed by salting-out, and one or more chromatography steps, including aqueous ion exchange chromatography, size exclusion chromatography steps, high pressure liquid chromatography, and affinity chromatography may be used to isolate the recombinant polypeptide. To produce a glycosylated peptide, or variant thereof, it is preferred that recombinant techniques be used. To produce a glycosylated peptide, or variant thereof, it is preferred that mammalian cells such as, COS-7 and Hep-G2 cells be employed in the recombinant techniques.

Purification of Recombinantly Expressed Peptides

Peptides of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and high pressure liquid chromatography. Well known techniques for refolding proteins may be employed to regenerate active conformation when the peptide is denatured during isolation and or purification.

Nucleic Acid Molecules

The terms “nucleic acid molecule”, “nucleic acid”, “nucleic acid sequence”, “nucleotide sequence”, “functional nucleic acid molecule” and “polynucleotide” are used interchangeably herein and refer to any nucleic acid molecule, DNA or RNA, which encodes a corresponding peptide according to the invention. These DNA or RNA molecules can also be present in vectors.

The present invention also provides isolated nucleic acid molecules that encode the VEGF-D or VEGF-C peptides of the present invention. The present nucleic acid molecules also encompass nucleic acid molecules having sequences that are capable of hybridizing under stringent conditions, preferably highly stringent conditions, to the nucleic acid molecules that encode the VEGF-D or VEGF-C peptides of the present invention. The person skilled in the art will appreciate that hybridization conditions are based on the melting temperature (Tm) of the nucleic acid molecule binding complex or probe.

The term “stringent conditions”, as used herein, is the “stringency” which occurs within a range from about Tm-5 to about Tm-20 (about 5° C. to about 20° C. below the melting temperature of the probe).

As used herein, “highly stringent” conditions employ at least 0.2×SSC buffer and at least 65° C. As recognized in the art, stringency conditions can be attained by varying a number of factors of the hybridization solution such as the length and nature, i.e., DNA or RNA, of the probe; the length and nature of the target sequence; the concentration of the salts; and the concentration of other components, such as formamide, dextran sulfate, and polyethylene glycol. All of these factors may be varied to generate conditions of stringency which are equivalent to the conditions listed above.

Nucleic acid molecules comprising sequences encoding a VEGF-D or VEGF-C peptide of the present invention may be synthesized in whole or in part using chemical methods or recombinant methods which are known in the art.

The nucleic acid molecules are useful for producing a VEGF-D or VEGF-C peptide. For example, an RNA molecule encoding a peptide may be used in a cell-free translation system to prepare such peptide. Alternatively, a DNA molecule encoding a VEGF-D or VEGF-C peptide may be introduced into an expression vector and used to transform cells. Suitable expression vectors include, for example, chromosomal, non-chromosomal and synthetic DNA sequences, e.g., derivatives of SV40, bacterial plasmids, phage DNAs, yeast plasmids, vectors derived from combinations of plasmids and phage DNAs, viral DNA such as vaccinia, adenovirus, fowl pox virus, pseudorabies, baculovirus, and retrovirus. The DNA sequence may be introduced into the expression vector by conventional procedures known in the art.

Accordingly, the present invention also relates to recombinant constructs comprising a nucleic acid molecule of the present invention.

Suitable constructs include, for example, vectors, such as a plasmid, phagemid, or viral vector, into which a sequence that encodes VEGF-D or VEGF-C peptide has been inserted. In the expression vector, the DNA sequence which encodes the peptide is operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator.

The recombinant expression vectors may also include an origin of replication and a selectable marker, such as the ampicillin resistance gene of E. coli to permit selection of transformed cells, i.e., cells that are expressing the heterologous DNA sequences. The nucleic acid molecule encoding the VEGF-D or VEGF-C peptide may be incorporated into the vector in frame with translation initiation and termination sequences.

The nucleic acid molecule encoding the VEGF-D or VEGF-C peptide can be used to express recombinant peptide using techniques well known in the art. A nucleic acid molecule encoding the VEGF-D or VEGF-C peptide of the invention may also be used to immunize animals.

The nucleic acid molecule of the invention also relates to a DNA sequence that can be derived from the amino acid sequence of the peptide of the invention bearing in mind the degeneracy of codon usage. This is well known in the art, as is knowledge of codon usage in different expression hosts, which is helpful in optimizing the recombinant expression of the peptide of the invention.

The invention also provides nucleic acid molecules which are complementary to all the above described nucleic acid molecules.

When the nucleic acid molecules of the invention are used for the recombinant production of peptides of the present invention, the nucleic acid molecule may include the coding sequence for the peptide by itself or the coding sequence for the peptide in reading frame with other coding sequences, such as those encoding a leader or secretory sequence, a pre-, or pro- or prepro-protein sequence, or other fusion peptide portions. For example, a marker sequence which facilitates purification of the fused peptide can be encoded. In certain embodiments of this aspect of the invention, the marker sequence is a hexa-histidine peptide, as provided in the pQE vector (Qiagen, Inc.), or is an HA tag, or is glutathione-S-transferase, or is a FLAG octapeptide.

The nucleic acid molecule may also contain non-coding 5′ and 3′ sequences, such as transcribed, non-translated sequences, splicing and polyadenylation signals, ribosome binding sites and sequences that stabilize mRNA.

Vectors, Host Cells, Expression

The present invention also relates to vectors which comprise a nucleic acid molecule of the present invention, and host cells which are genetically engineered with vectors of the invention and to the production of peptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for nucleic acid molecules of the present invention. Introduction of nucleic acid molecules into host cells can be effected by one skilled in the art using methods such as calcium phosphate transfection, DEAE-dextran mediated transfection, transfection, microinjection, cationic lipid-mediated transfection, electroporation, transduction, scrape loading, ballistic introduction or infection.

Representative examples of appropriate hosts include bacterial cells, such as meningococci, streptococci, staphylococci, E. coli, Streptomyces and Bacillus subtilis cells; fungal cells and Aspergillus cells; yeast cells; insect cells such as Drosophila S2 and Spodoptera Sf9 cells; animal cells such as CHO, COS, HeLa, C127, 3T3, BHK, HEK293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression.

Generally, any system or vector suitable to maintain, propagate or express nucleic acid molecules to produce a peptide in a host may be used. The appropriate nucleotide sequence may be inserted into an expression system by any of a variety of well known and routine techniques.

For secretion of the translated peptide into the lumen of the endoplasmic reticulum, into the periplasmic space, or into the extracellular environment, appropriate secretion signals may be incorporated into the desired peptide. These signals may be endogenous to the peptide or they may be heterologous signals.

Antibodies

The peptides of the invention, or cells expressing them, can also be used as immunogens to produce antibodies that specifically bind the wild-type VEGF-D or VEGF-C.

An “immunogen” refers to substance which is capable of eliciting (inducing) an immune response. “Immunogenic” or “immunogenicity” refers to the corresponding immune response thus elicited. The terms “immunogen”, “antigen”, “epitope”, and “vaccine” may be used interchangeably herein.

Similarly, the term “specific binding”, “specifically binds” or “binds specifically” refers to binding where a molecule binds to a particular epitope on a particular polypeptide without substantially binding to any other polypeptide or polypeptide epitope. Such binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. As used herein, “specific binding” is used in relation to the interaction between the molecular components of VEGF-D or VEGF-C activity, including receptors for VEGF-D and VEGF-C. Specific binding is also used in relation to the interaction between the molecular components of VEGF-D or VEGF-C activity and agents that partially or fully block, neutralize, reduce or antagonize a biological activity of VEGF-D or VEGF-C. In particular, specific binding refers to a molecule having a K_{d} at least 2-fold greater than that of a non-specific target, preferably a molecule having a Kd at least 4-fold, 6-fold, 8-fold, 10-fold, or greater than that of a non-specific target. Alternatively, specific binding can be expressed as a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or less.

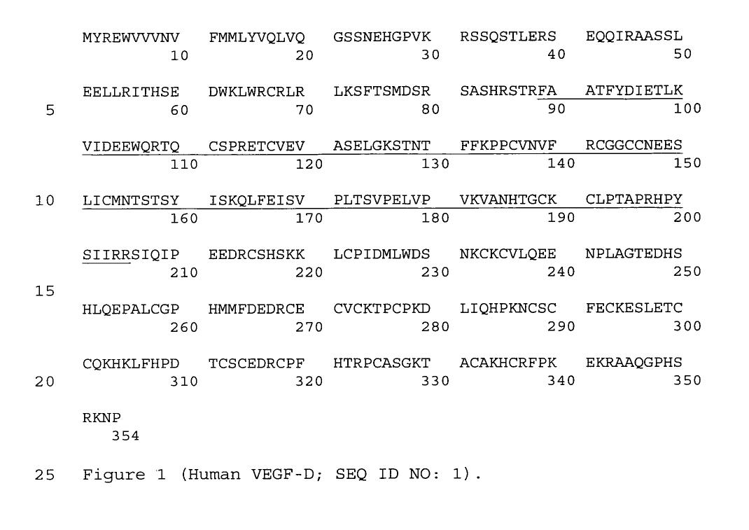

As used herein, “binds specifically to VEGF-D” refers to specific binding to the mature, fully processed form of VEGF-D comprising the VHD of VEGF-D underlined in FIG. 1.

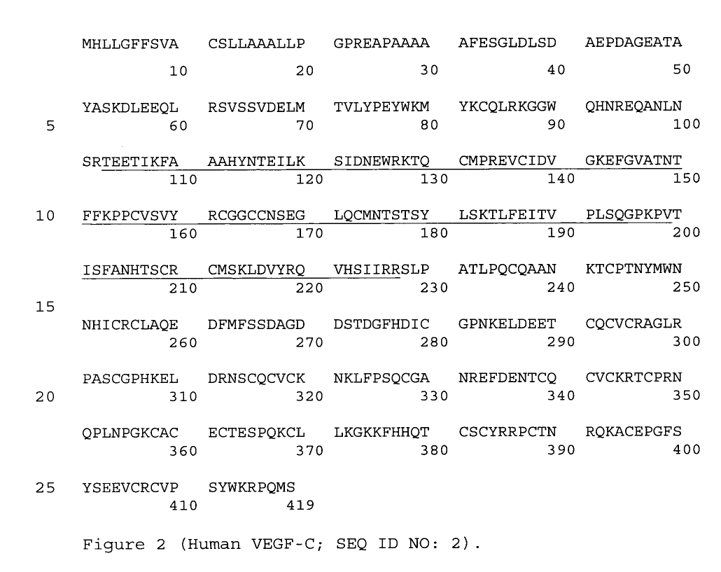

As used herein, “binds specifically to VEGF-C” refers to specific binding to the mature, fully processed form of VEGF-C comprising the VHD of VEGF-C underlined in FIG. 2.

Antibodies generated against the peptide of the invention can be obtained by administering the peptide to an animal using routine protocols in the immunization of an animal with an antigen, the collection of the blood, the isolation of the serum and the use of the antibodies that react with the peptide. The serum or immunoglobulin fraction containing the antibodies may be used in analyzing the protein. For preparation of monoclonal antibodies, any technique which provides antibodies produced by continuous cell line cultures can be used.

Examples include the hybridoma technique, the trioma technique, the human B-cell hybridoma technique, and the EBV-hybridoma technique.

Techniques for the production of single chain antibodies can also be adapted to produce single chain antibodies to peptides of this invention. Also, transgenic mice, or other organisms including other mammals, may be used to express humanized antibodies.

The term “antibody” is used in the broadest sense and specifically covers, for example, polyclonal antibodies, monoclonal antibodies (including antagonist and neutralizing antibodies), antibody compositions with polyepitopic specificity, single chain antibodies, and fragments of antibodies, provided that they exhibit the desired biological or immunological activity. “Antibody” may also be used interchangeably with “immunoglobulin”.

An “antibody inhibitor” will specifically bind to a particular polypeptide without substantially binding to any other polypeptide or polypeptide epitope. Such binding will partially or fully block, neutralize, reduce or antagonize VEGF-D or VEGF-C activity. Such target molecules include VEGFR-2, VEGFR-3, VEGF-C and VEGF-D, for example.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. Generally, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS PAGE under reducing or non-reducing conditions using Coomassie blue or, preferably, silver stain. An isolated antibody includes the antibody in situ within recombinant cells since at least one component of the antibody's natural environment will not be present. Ordinarily, however, an isolated antibody will be prepared by at least one purification step.

Polyclonal Antibodies

Polyclonal antibodies may be raised in animals by multiple subcutaneous (s.c.) or intraperitoneal (i.p.) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or RN═C═NR¹, where R and R¹ are different alkyl groups.

Animals may be immunized against the antigen, immunogenic conjugates, or derivatives by combining the antigen, conjugate or derivative with three volumes of Freund's complete adjuvant and injecting the solution intradermally at multiple sites. One month later, the animals may be boosted with ⅕ to 1/10 the original amount of peptide or conjugate in Freund's complete adjuvant by subcutaneous injection at multiple sites. Seven to 14 days later, the animals may be bled and the serum assayed for antibody titer. Animals may be boosted until the titer plateaus. Conjugates also can be made in recombinant cell culture as protein fusions. Also, aggregating agents such as alum are suitably used to enhance the immune response.

Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method.

Monoclonal antibodies may be made using the hybridoma method in which a mouse or other appropriate host animal, such as a hamster, is immunized to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium, which medium may contain one or more substances that inhibit the growth or survival of the unfused, parental myeloma cells (also referred to as fusion partner).

Culture medium in which hybridoma cells are growing is assayed for production of monoclonal antibodies directed against the antigen. The binding specificity of monoclonal antibodies produced by hybridoma cells may be determined by immunoprecipitation or by an in vitro binding assay, such as radioimmunoassay (RIA) or enzyme-linked immunosorbent assay (ELISA).

Once hybridoma cells that produce antibodies of the desired specificity, affinity, and/or activity are identified, the clones may be subcloned by limiting dilution procedures and grown by standard methods. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxyapatite chromatography, gel electrophoresis, dialysis.

DNA encoding the monoclonal antibodies is readily isolated and sequenced using conventional procedures. The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

Monoclonal antibodies or antibody fragments can be isolated from antibody phage libraries. High affinity (nM range) human antibodies can be generated by chain shuffling, as well as combinatorial infection and in vivo recombination as a strategy for constructing very large phage libraries. Thus, these techniques are viable alternatives to traditional monoclonal antibody hybridoma techniques for isolation of monoclonal antibodies.

The DNA that encodes the antibody may be modified to produce chimeric or fusion antibody polypeptides. The monoclonal antibodies used herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

Human and Humanized Antibodies

The anti-VEGF-D or anti-VEGF-C antibodies of the invention may comprise humanized antibodies or human antibodies. “Humanized” forms of non human (e.g., rodent) antibodies are chimeric antibodies that contain minimal sequence derived from the non human antibody. Humanized antibodies are chimeric antibodies, antibody chains or fragments thereof (such as Fv, Fab, Fab′, F(ab′)₂, or other antigen-binding subsequences of antibodies). Humanized antibodies include human antibodies (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human antibody are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human antibody and all or substantially all of the FR regions are those of a human antibody consensus sequence. The humanized antibody optimally also will comprise at least a portion of an antibody constant region (Fc), typically that of a human antibody.

Methods for humanizing non-human antibodies are well known in the art. Generally, a humanized antibody has one or more amino acid residues introduced into it from a source which is non-human.

Various forms of humanized anti-VEGF-D or anti-VEGF-C antibodies are contemplated. For example, the humanized antibody may be an antibody fragment, such as a Fab. Alternatively, the humanized antibody may be an intact antibody, such as an intact IgG₁ antibody.

Various humanization strategies have been described in the prior art and it is envisaged that practice of the invention extends to the use of both known humanization strategies and any new strategies to be developed in the future.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production.

Alternatively, phage display technology can be used to produce human antibodies and antibody fragments in vitro, from immunoglobulin variable (V) domain gene repertoires from unimmunized donors. Phage display can be performed in a variety of formats. Several sources of V-gene segments can be used for phage display.

Antibody Fragments

“Antibody fragments” comprise a portion of an antibody, preferably the antigen binding or variable region of the intact antibody. Examples of antibody fragments include Fab, Fab′, F(ab′)₂, and Fv fragments; diabodies; linear antibodies; single chain antibody molecules; and multispecific antibodies formed from antibody fragments.

Papain digestion of antibodies produces two identical antigen binding fragments, called “Fab” fragments, and a residual “Fc” fragment, a designation reflecting the ability to crystallize readily. Each Fab fragment is monovalent with respect to antigen binding, i.e., it has a single antigen binding site. Pepsin treatment of an antibody yields a single large F(ab′)₂ fragment which roughly corresponds to two disulfide linked Fab fragments having divalent antigen binding activity and is still capable of cross linking antigen. Fab′ fragments differ from Fab fragments by having additional residues at the carboxy terminal of the C_{H}1 domain including one or more cysteines from the antibody hinge region. Fab′ SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen recognition binding site. This fragment consists of a dimer of one heavy and one light chain variable region domain in tight, non covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs that specifically bind an antigen) has the ability to recognize and bind antigen, although at a lower affinity than the entire binding site.

“Single chain Fv” abbreviated as “scFv” are antibody fragments that comprise the V_{H} and V_{L} antibody domains connected into a single polypeptide chain. Preferably, the scFv polypeptide further comprises a polypeptide linker between the V_{H} and V_{L} domains which enables the scFv to form the desired structure for antigen binding.

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues also may be used.

Other techniques for the production of antibody fragments will be apparent to the skilled practitioner. The antibody of choice is a scFv fragment. Fv and scFv are the only species with intact combining sites that are devoid of constant regions; thus, they are suitable for reduced nonspecific binding during in vivo use. The antibody fragment may also be a “linear antibody”, which may be monospecific or bispecific.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence.

Bispecific Antibodies

Bispecific antibodies are antibodies that have binding specificities for at least two different epitopes. A bispecific antibody may bind to VEGF-D and VEGF-C. Bispecific antibodies can be prepared as full length antibodies or antibody fragments (e.g., F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities.

According to a different approach, antibody variable domains with the desired binding specificity (antibody-antigen combining sites) are fused to immunoglobulin constant domain sequences.

Bispecific antibodies include cross-linked or “heteroconjugate” antibodies. Heteroconjugate antibodies are composed of two covalently joined antibodies. It is contemplated that the antibodies may be prepared in vitro using known methods in synthetic protein chemistry, including those involving crosslinking agents. For example, one of the antibodies in the heteroconjugate can be coupled to avidin, the other to biotin. Heteroconjugate antibodies may be made using any convenient cross-linking methods. Suitable cross-linking agents and cross-linking techniques are well known in the art.

Techniques for generating bispecific antibodies from antibody fragments have also been described in the literature. For example, bispecific antibodies can be prepared using chemical linkage.

Recent progress has facilitated the direct recovery of Fab′-SH fragments from E. coli, which can be chemically coupled to form bispecific antibodies. Various techniques for making and isolating bispecific antibody fragments directly from recombinant cell culture have also been described.

The term “diabodies” refers to small antibody fragments prepared by constructing scFv fragments with short linkers (about 5 to 10 residues) between the V_{H} and V_{L} domains such that inter chain but not intra chain pairing of the V domains is achieved, resulting in a bivalent fragment, i.e., fragment having two antigen binding sites. Bispecific diabodies are heterodimers of two “crossover” scFv fragments in which the V_{H} and V_{L} domains of the two antibodies are present on different polypeptide chains.

According to an alternative “diabody” technology for making bispecific antibody fragments, the fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of scFv dimers has also been reported.

Antibodies with more than two valencies are contemplated for use in the invention. For example, trispecific antibodies can be prepared.

Multivalent Antibodies

A multivalent antibody may be internalized (and/or catabolized) faster than a bivalent antibody by a cell expressing an antigen to which the antibodies bind. The antibodies used in the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight antigen binding sites. The multivalent antibody comprises at least one polypeptide chain (and may comprise two polypeptide chains), wherein the polypeptide chain(s) comprise two or more variable domains. For instance, the polypeptide chain(s) may comprise VD₁-(X₁)ₙ-VD₂-(X₂)ₙ-Fc, wherein VD₁ is a first variable domain, VD₂ is a second variable domain, Fc is one polypeptide chain of an Fc region, X₁ and X₂ represent an amino acid or polypeptide, and n is 0 or 1. For instance, the polypeptide chain(s) may comprise: V_{H}-C_{H}1-flexible linker-V_{H}-C_{H}1-Fc region chain; or V_{H}-C_{H}1-V_{H}-C_{H}1-Fc region chain. The multivalent antibody herein may, for instance, comprise from about two to about eight light chain variable domain polypeptides. The light chain variable domain polypeptides contemplated here comprise a light chain variable domain and, optionally, further comprise a C_{L} domain.

The above-described antibodies may be employed to isolate or to identify clones expressing the peptide or to purify the peptides of the invention by affinity chromatography. Such antibodies also may be employed in analytical or diagnostic methods to detect peptides, polypeptides or proteins, of variants of fragments thereof, comprising the peptides according to the invention.

Formulation for Administration

The peptide, nucleic acid molecule, antibody or vaccine may be provided as a pharmaceutical composition or veterinary composition.

A “pharmaceutical composition” is one which is suitable for administration to humans. A “veterinary composition” is one that is suitable for administration to animals. “Composition” is used interchangeably with “formulation” herein.

The pharmaceutical or veterinary compositions used in the methods of the invention may comprise a pharmaceutically acceptable carrier and optionally another therapeutic agent. Each carrier, diluent, adjuvant and/or excipient must be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained.

Similarly, a “pharmaceutically acceptable” salt or ester is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

The pharmaceutical composition may be administered orally, topically, or parenterally in formulations containing conventional non-toxic pharmaceutically acceptable carriers, adjuvants, and vehicles.

The term “parenteral” as used herein includes intravenous, intraarterial, intraperitoneal, intramuscular, subcutaneous, subconjunctival, intracavity, transdermal and subcutaneous injection, aerosol for administration to lungs or nasal cavity or administration by infusion by, for example, osmotic pump.

Pharmaceutical Compositions

Pharmaceutical compositions which comprise mixtures of VEGF-D and/or VEGF-C peptides, nucleic acid molecules that encode the same, or antibodies generated by the peptides of the invention are preferably formulated for use as a pharmaceutical composition (e.g., an immunogen or a vaccine). Such pharmaceutical compositions generally comprise further a pharmaceutically acceptable carrier, excipient, or diluent.

In addition to the peptides (which function as antigens) or the nucleic acid molecules which encode the same, other components, such as a vehicle for antigen delivery or an adjuvant may be included in the pharmaceutical composition.

Veterinary Compositions

The peptide, nucleic acid molecule or antibody of the invention may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue, particularly adapted for protection through the rumen if to be administered to ruminants;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, e.g. as a cream, ointment or spray applied to the skin; or

(d) intravaginally, e.g. as a pessary, cream or foam.

Vaccines

A further aspect of the invention relates to an immunogenic formulation or vaccine formulation comprising an immunogenic amount of at least one peptide of the invention which, when introduced into a mammalian host, induces an immunological response in that mammal to VEGF-D or VEGF-C peptide or both VEGF-D and VEGF-C peptides, wherein the composition comprises a nucleic acid molecule encoding the VEGF-D or VEGF-C peptide itself or the VEGF-D and VEGF-C peptides themselves. The vaccine formulation may further comprise a suitable carrier. The VEGF-D or VEGF-C vaccine formulation may be administered orally, intranasally or parenterally (including subcutaneous, intramuscular, intravenous, intradermal, transdermal injection).

The terms “vaccine”, “immunogen”, “vaccine formulation”, and “immunogenic formulation” are used herein interchangeably. A vaccine is included in the class of a pharmaceutical composition or a veterinary composition.

The vaccine may be directed against cancerous diseases associated with VEGF-D or VEGF-C or both VEGF-D and VEGF-C, which is characterized in that it is recognized immunologically by the VEGF-D neutralizing antibody or an antibody according to the invention. Alternatively, the vaccine may be directed against a condition responsive to neutralizing VEGF-D, VEGF-C, or both VEGF-D and VEGF-C. The condition may include dysregulated angiogenesis, dysregulated lymphangiogenesis, rheumatoid arthritis, psoriasis, lymphangiolieomyomatosis, and other inflammatory conditions. The vaccine comprises at least one peptide according to the invention and/or a functional nucleic acid molecule for producing said peptide.

Carriers and Excipients

In one embodiment, the vaccine also comprises a pharmaceutically acceptable excipient. In one embodiment, the vaccine also comprises a carrier, such that the peptide or its functional variant is conjugated to the immunogenic carrier. Any immunogenic carrier that does not endanger human health can be used. Such carriers can be macromolecules of any kind, it being important that a selected carrier is nontoxic to animals and in particular to humans and involves no dangers e.g. of a phage or phage particle with respect to any contained toxins or the possibility of infection e.g. of intestinal bacteria, and is nonpoisonous and does not trigger any serum sicknesses or food allergies. Conjugation to a carrier has the consequence of increasing the immunogenicity of the vaccine.

Examples of carriers that might be used are keyhole limpet hemocyanin (KLH), tetanus toxoid (TT), albumen-binding protein (ABP) or bovine serum albumen (BSA).

It may also be useful to conjugate the peptide to a targeting molecule or construct which directs delivery of the peptide to a site at which an immune response is desired. For example, conjugating the peptide to a molecule or construct which directed the peptide to a tumor, for example a solid tumor, may be particularly beneficial. This could result in a stronger localised immune response and provide a more effective therapeutic response.

Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or antibodies; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

Oral excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Although any suitable carrier known to those of ordinary skill in the art may be employed in the pharmaceutical compositions of this invention, the type of carrier will vary depending on the mode of administration and whether a substantial release is desired. For parenteral administration, such as subcutaneous injection, the carrier preferably comprises water, saline, alcohol, a fat, a wax, or a buffer.

Biodegradable microspheres (e.g., polylactic galactide) may also be employed as carriers for the pharmaceutical compositions of this invention. Optionally, the pharmaceutical composition comprises an adjuvant.

The VEGF-D or VEGF-C peptide mixtures and the nucleic acid molecules that encode the same are useful for enhancing or eliciting, in a subject, a humoral response and, preferably, a cellular immune response (e.g., the generation of antigen-specific cytolytic T cells).

Conjugation

Conjugation of the peptides or their variants to the carrier can be done in any way, for example by genetic engineering or by chemical means, i.e. carrier and functional group are linked by a chemical reaction. By genetic engineering the protein carrier molecule can be coupled with the peptide or its variant by inserting a DNA or RNA sequence coding for the total sequence of the conjugate into an expression system by which the total conjugate is then expressed. This form of conjugation can of course only be applied if the total conjugate is a protein molecule.

In one embodiment, the peptides or their variants are conjugated to the carrier by chemical means. That is, the linkage of peptide or its variant and the carrier to the conjugate is effected by chemical means.

The peptides or their functional variants can be conjugated to the carrier as mono-, di-, tri- or oligomer.

If the conjugation of a di- or oligomeric peptide conjugate is performed using a genetic engineering method, the DNA or RNA portions coding for the peptides are integrated lined up one after the other once or several times into the DNA or RNA sequence coding for the carrier. This obtains the expression of di- or oligomeric peptide conjugates.

The mono- or oligomers of the peptides or their functional variants can be conjugated to the carrier both in single and in multiple form, i.e. one or more peptide molecules or their functional variants are attached to a carrier.

Linkers

The vaccine of the invention may also comprise a non-specific linker that can be present between the peptide sequence and the immunogenic carrier and is preferably joined to the peptide sequence or co-synthesized, whether chemically or by genetic engineering, to facilitate coupling to the carrier such as keyhole limpet hemocyanin (KLH), tetanus toxoid (TT), albumen-binding protein (ABP) or bovine serum albumen (BSA), and/or to serve as spacers between the peptide sequence and the carrier.

Adjuvants

The vaccine of the invention may further comprise an adjuvant.

An “adjuvant” refers to an immunostimulatory substance designed to enhance the immunogenicity of an antigen, epitope, or peptide.

Suitable adjuvants include an aluminium salt such as aluminum hydroxide gel (alum) or aluminum phosphate, but may also be a salt of calcium, iron or zinc, or may be an insoluble suspension of acylated tyrosine, or acylated sugars, cationically or anionically derivatized polysaccharides, or polyphosphazenes. Other known adjuvants include CpG containing oligonucleotides. The oligonucleotides are characterized in that the CpG dinucleotide is unmethylated.

Further preferred adjuvants are those which induce an immune response preferentially of the Th1-type. High levels of Th1-type cytokines tend to favor the induction of cell mediated immune responses to the given antigen, whilst high levels of Th2-type cytokines tend to favor the induction of humoral immune responses to the antigen. Suitable adjuvant systems include, for example monophosphoryl lipid A, preferably 3-de-O-acylated monophosphoryl lipid A (3D-MPL), or a combination of 3DMPL together with an aluminium salt. CpG oligonucleotides also preferentially induce a Th1 response.

An enhanced system involves the combination of a monophosphoryl lipid A and a saponin derivative particularly the combination of QS21 and 313-MPL, or a less reactogenic composition where the QS21 is quenched with cholesterol. A particularly potent adjuvant formulation involves QS21 3D-MPL & tocopherol in an oil-in-water emulsion.

Preparation

The peptides or functional peptide variants may be prepared synthetically by chemical means. This may be done with the aid of solid phase synthesis. Similarly, the vaccine can be produced in diverse ways by genetic engineering or chemical means. If chemical means are used, solid phase peptide synthesis is expedient.

An example of a genetic engineering production method is manipulation of microorganisms such as E. coli. These are manipulated so that they express the peptides as such or the total conjugates consisting of peptide and carrier coupled thereto.

Administration

The vaccine can be administered in different ways. The vaccines containing the peptides themselves or their functional peptide variants can be administered for example intravenously, subcutaneously or else by oral taking of the vaccine in capsule or tablet form. If the vaccine contains functional nucleic acid variants of the peptides, administration can also be done using an ex vivo procedure, which comprises removal of cells from an organism, penetration of the vaccine into these cells, and repenetration of the treated cells into the organism.

The peptide, nucleic acid molecule, antibody, vaccine or composition of the invention may be administered orally as tablets, aqueous or oily suspensions, lozenges, troches, powders, granules, emulsions, capsules, syrups or elixirs. The composition for oral use may contain an agent selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

Examples of vehicles for antigen delivery include aluminium salts, water-in-oil emulsions, biodegradable oil vehicles, oil-in-water emulsions, biodegradable microcapsules, and liposomes. For the vaccines that comprise the peptide, one potential vehicle for antigen delivery is a biodegradable microsphere, which preferably is comprised of poly(D,L-lactide-co-glycolide) (PLGA).

Formulations or compositions for parenteral administration include sterile aqueous or non-aqueous solutions, suspensions, and emulsions. Aqueous and non-aqueous sterile injection solutions may contain anti-oxidants, buffers, bacteriostats, and solutes, which render the formulation isotonic with the blood of the recipient, and aqueous and non-aqueous sterile suspensions, which may include suspending agents or thickening agents.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The compositions may also contain other active compounds providing supplemental, additional, or enhanced therapeutic functions.

Other therapeutically useful agents, such as growth factors (e.g., BMPs, TGF-P, FGF, IGF), cytokines (e.g., interleukins and CDFs), antibiotics, and any other therapeutic agent beneficial for the condition being treated may optionally be included in or administered simultaneously or sequentially with the peptide, nucleic acid molecule, or antibody of the invention. Other agents that may be effective for those purposes include tumor necrosis factor (TNF), an antibody capable of inhibiting or neutralizing the angiogenic activity of acidic or basic fibroblast growth factor (FGF) or hepatocyte growth factor (HGF), an antibody capable of inhibiting or neutralizing the coagulant activities of tissue factor, protein C, or protein S, an antibody capable of binding to HER2 receptor, or one or more conventional therapeutic agents such as, for example, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Such other agents may be present in the composition being administered or may be administered separately. Also, the agent is suitably administered serially or in combination with radiological treatments, whether involving irradiation or administration of radioactive substances.

In one embodiment, vascularization of tumors is attacked in combination therapy. The agent of the invention may be administered to tumor-bearing patients at therapeutically effective doses as determined for example by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein, or heat or radiation.

Since the auxiliary agents will vary in their effectiveness, it is desirable to compare their impact on the tumor by matrix screening in conventional fashion. The administration of an agent of the invention, such as an antibody, and TNF is repeated until the desired clinical effect is achieved. Alternatively, the anti-VEGF agent may be administered together with TNF and, optionally, auxiliary agent(s). In instances where solid tumors are found in the limbs or in other locations susceptible to isolation from the general circulation, the therapeutic agents described herein are administered to the isolated tumor or organ. In other embodiments, a FGF or platelet-derived growth factor (PDGF) antagonist, such as an anti-FGF or an anti-PDGF neutralizing antibody, is administered to the patient in conjunction with the agent of the invention. Treatment with an agent of the invention may be suspended during periods of wound healing or desirable neovascularization.

An agent of the invention can be administered alone or in combination with one or more additional therapies such as chemotherapy radiotherapy, immunotherapy, surgical intervention, or any combination of these. Long-term therapy is equally possible as is adjuvant therapy in the context of other treatment strategies.

Antibodies of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering antibodies of the invention that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides methods and compositions for the specific destruction of cells (e.g., the destruction of tumor cells) by administering antibodies of the invention in association with toxins or cytotoxic prodrugs. In specific embodiments, the invention provides compositions and in vitro or in vivo methods for the specific destruction of cells expressing a VEGF-D/VEGF-C receptor by contacting VEGF-D/VEGF-C receptor-expressing cells with antibodies of the invention in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

In certain embodiments, therapeutic agents of the invention can be used alone. Alternatively, the agents may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject polypeptide therapeutic agent.

A wide array of conventional compounds has been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplatic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a therapeutic agent of the present invention is administered in combination with another conventional anti-neoplastic agent, either concomitantly or sequentially, such therapeutic agent may be found to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Certain chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (e.g., VEGF inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as an anti-bFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. In addition, there are a wide variety of compounds that can be used to inhibit angiogenesis, for example, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naladase inhibitors.

Administration “in combination with” a further therapeutic agent includes simultaneous (concurrent) and consecutive administration in any order.

It is especially advantageous to formulate the veterinary or pharmaceutical compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Alternatively, the compositions may be presented in multi-dose form.

Examples of dosage units include sealed ampoules and vials and may be stored in a freeze-dried condition requiring only the addition of the sterile liquid carrier immediately prior to use.

The compositions may also be included in a container, pack, or dispenser together with instructions for administration.

Dosages and desired drug concentrations of pharmaceutical or veterinary compositions of the present invention may vary depending on the particular use envisioned. The determination of the appropriate dosage or route of administration is well within the skill of an ordinary physician.

When in vivo administration of a peptide, nucleic acid molecule, or antibody of the invention is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature. It is anticipated that different formulations will be effective for different treatment compounds and different disorders, that administration targeting one organ or tissue, for example, may necessitate delivery in a manner different from that to another organ or tissue.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Where sustained-release administration of a peptide, nucleic acid molecule, or antibody of the invention is desired in a formulation with release characteristics suitable for the treatment of cancer, for example, requiring administration of the peptide, nucleic acid molecule, or antibody of the invention, microencapsulation of the peptide, nucleic acid molecule, or antibody of the invention is contemplated. The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Microencapsulation of recombinant proteins for sustained release has been successfully performed with human growth hormone (rhGH), interferon- (rhIFN-), interleukin-2, and MN rgp120.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition.

If the VEGF-D or VEGF-C activity target is intracellular and whole antibodies are used to treat a condition, internalizing antibodies are preferred. However, lipofections or liposomes can also be used to deliver the antibody or antibody fragment into cells.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The formulation herein may also contain more than one active compound as necessary for the particular indication being treated, preferably those with complementary activities that do not adversely affect each other. Alternatively, or in addition, the composition may comprise an agent that enhances its function. Such molecules are suitably present in combination in amounts that are effective for the purpose intended.

Methods and Uses

The present invention also provides a method of treating a cancer which is associated with over-expression of VEGF-D or VEGF-C or both VEGF-D and VEGF-C and also relates to uses of the peptide, nucleic acid molecule, or antibody according to the invention. Alternatively, the invention provides a method of treating a condition responsive to neutralizing VEGF-D, VEGF-C, or both VEGF-D and VEGF-C. In one embodiment, the condition may include dysregulated angiogenesis, dysregulated lymphangiogenesis, rheumatoid arthritis, psoriasis, lymphangiolieomyomatosis, and other inflammatory conditions.

“Treating” or “treatment” refers to both therapeutic treatment and prophylactic or preventative measures, wherein the aim is to prevent, ameliorate, reduce or slow down (lessen) cancer.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The “subject” includes a mammal. The mammal may be a human, or may be a domestic, zoo, or companion animal. While it is particularly contemplated that the methods of the invention are suitable for medical treatment of humans, they are also applicable to veterinary treatment, including treatment of companion animals such as dogs and cats, and domestic animals such as horses, cattle and sheep, or zoo animals such as felids, canids, bovids, and ungulates. A subject may be afflicted with cancer or other disorder, or may not be afflicted with cancer or other disorder (i.e., free of detectable disease).

The method comprises administering to a subject a therapeutically effective amount of a pharmaceutical or veterinary composition for inducing an immunological response in a mammal comprising a VEGF-D and/or VEGF-C peptide mixture adequate to produce antibody to inhibit VEGF-D and/or VEGF-C, and/or comprising an antibody that binds specifically to and neutralizes VEGF-D and/or VEGF-C of the present invention.

The term “therapeutically effective amount” refers to an amount of the agent capable of reducing VEGF-D or VEGF-C activity in a subject or mammal to a level which is beneficial to treat cancer or other condition. A therapeutically effective amount may be determined empirically and in a routine manner in relation to treating cancer or other condition, and will result in increased life expectancy.

In yet another embodiment, the invention relates to a method of inducing immunological response in a mammal which comprises, delivering a peptide of the invention via a vector directing expression of a nucleic acid molecule of the invention in vivo in order to induce such an immunological response to produce antibody to protect said animal from diseases.

The present invention also provides methods of inhibiting angiogenesis in rapidly growing tissues and to inhibit growth of tumors in a subject.

An agent according to the invention may be useful in the treatment of various neoplastic and non-neoplastic diseases and disorders. Thus, the present invention provides a method of treating an angiogenesis-related disease and/or disorder described herein or otherwise known in the art, comprising administering to an individual in need thereof a therapeutically effective amount of an agent of the invention.

Neoplasms and related conditions, cancers, tumors, malignant and metastatic conditions, tissues and organs which can be treated with an agent of the invention include, but are not limited to, abnormal vascular proliferation associated with phakomatoses, advanced malignancies, arrhenoblastomas, astrocytoma, biliary tract, bladder, blood born tumors such as leukemias, brain, breast, cavernous hemangioma, cervix, choriocarcinoma, colon, colorectal, edema (such as that associated with brain tumors), endometriosis, endometrium, esophagus, fibrosarcomas, gastric carcinomas, glioblastoma, head and neck cancer, hemangioblastoma, hemangioma, hepatoblastoma, Kaposi's sarcoma, kidney, larynx, leiomyosarcoma, liver, lung, medulloblastoma, Meigs' syndrome, melanoma, nasopharyngeal carcinoma, neuroblastomas, non-small cell lung cancer, oligodendroglioma, osteogenic sarcoma, ovarian, pancreas, parotid, primary tumors and metastases, prostate, rectum, renal cell, retinoblastoma, rhabdomyosarcoma, Schwannoma, skin, solid tumors, stomach, testes, thecomas, thyroid, urinary tract, uterus, and Wilm's tumor.

In one embodiment, the antibodies may be delivered topically, in order to treat cancers such as skin cancer, head and neck tumors, breast tumors, and Kaposi's sarcoma. In other embodiments, antibodies may be utilized to treat superficial forms of bladder cancer by, for example, intravesical administration.

An agent such as an antibody may be delivered directly into the tumor, or near the tumor site, via injection or a catheter. Of course, as the person skilled in the art will appreciate, the appropriate mode of administration will vary according to the cancer to be treated.

An agent of the invention may be useful in treating other disorders, besides cancers, which involve angiogenesis. Non-neoplastic conditions that are amenable to treatment include acoustic neuromas, age-related macular degeneration, angiofibroma, arteriovenous malformations, artheroscleric plaques, ascites, atherosclerosis, benign tumors, cerebral collaterals, chronic inflammation, corneal graft rejection and other tissue transplantation rejection, coronary collaterals, Crohn's disease, delayed wound healing, diabetic and other proliferative retinopathies, endometriosis, fibromuscular dysplasia, granulations, hemangiomas, hemophiliac joints, hypertrophic scars (keloids), ischemic limb angiogenesis, lung inflammation, macular degeneration, myocardial angiogenesis, neovascular glaucoma, nephrotic syndrome, neurofibromas, nonunion fractures, ocular angiogenic diseases, Osler-Webber Syndrome, pericardial effusion (such as that associated with pericarditis), plaque neovascularization, pleural effusion, preeclampsia, psoriasis, pyogenic granulomas, retinoblastoma, retinopathy of prematurity, retrolental fibroplasia, rheumatoid arthritis, rubeosis, scleroderma, telangiectasia, thyroid hyperplasias (including Grave's disease), trachoma, uvietis and Pterygia (abnormal blood vessel growth) of the eye, vascular adhesions, vasculogenesis, and wound granulation.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the agent of the present invention is expected to be especially useful in reducing the severity of AMD.

In one embodiment of the present invention, methods are provided for treating hypertrophic scars and keloids, comprising the step of administering antibodies of the invention to a hypertrophic scar or keloid. Antibodies of the invention may be injected directly into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development.

Ocular disorders associated with neovascularization which can be treated with an agent of the present invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization.

Thus, one embodiment of the present invention provides a method for treating neovascular diseases of the eye such as corneal neovascularization (including corneal graft neovascularization), comprising the step of administering to a patient a therapeutically effective amount of an agent (including antibodies) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

In one embodiment, an antibody of the invention may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. The anti-angiogenic composition may be prepared with a muco-adhesive polymer which binds to cornea. The anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

The antibodies described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2 to 3 times per year. A steroid could also be added to the injection solution to reduce inflammation resulting from the injection itself.

An agent of the invention may be used for treating neovascular glaucoma, comprising the step of administering to a patient a therapeutically effective amount of an agent such as an antibody to the eye, such that the formation of blood vessels is inhibited. In one embodiment, the compound may be administered topically to the eye in order to treat early forms of neovascular glaucoma. Within other embodiments, the agent may be implanted by injection into the region of the anterior chamber angle. Within other embodiments, the agent may also be placed in any location such that the agent is continuously released into the aqueous humor. In another embodiment of the present invention, methods are provided for treating proliferative diabetic retinopathy, comprising the step of administering to a patient a therapeutically effective amount of a an antibody to the eyes, such that the formation of blood vessels is inhibited.

In one embodiment of the invention, proliferative diabetic retinopathy may be treated by injection into the aqueous humor or the vitreous, in order to increase the local concentration of the antibodies in the retina. Preferably, this treatment should be initiated prior to the acquisition of severe disease requiring photocoagulation.

In another embodiment of the present invention, methods are provided for treating retrolental fibroplasia, comprising the step of administering to a patient a therapeutically effective amount of an antibody to the eye, such that the formation of blood vessels is inhibited. The compound may be administered topically, via intravitreous injection and/or via intraocular implants.

Moreover, disorders and/or states, which can be treated, prevented, diagnosed, and/or prognosed with an agent such as an antibody of the invention include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one embodiment relating to a method of birth control, an amount of the agent sufficient to block embryo implantation is administered before or after intercourse and fertilization have occurred, thus providing an effective method of birth control, possibly a “morning after” method. Antibodies may also be used in controlling menstruation or administered as either a peritoneal lavage fluid or for peritoneal implantation in the treatment of endometriosis.

Antibodies of the present invention may be incorporated into surgical sutures in order to prevent stitch granulomas.

Antibodies may be utilized in a wide variety of surgical procedures. For example, within one embodiment of the present invention, a composition (in the form of, for example, a spray or film) may be utilized to coat or spray an area prior to removal of a tumor, in order to isolate normal surrounding tissues from malignant tissue, and/or to prevent the spread of disease to surrounding tissues. Within another embodiment of the present invention, compositions (e.g., in the form of a spray) may be delivered via endoscopic procedures in order to coat tumors, or inhibit angiogenesis in a desired locale. Within yet another embodiment of the present invention, surgical meshes which have been coated with a composition comprising an agent of the invention having anti-angiogenic activity, for example an anti-VEGFD and/or anti-VEGF-C antibody, may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

According to further embodiments of the present invention, methods are provided for treating tumor excision sites, comprising administering an agent of the invention such as an antibody to the resection margins of a tumor subsequent to excision, such that the local recurrence of cancer and the formation of new blood vessels at the site is inhibited. Within one embodiment of the invention, the anti-angiogenic agent, for example an antibody, is administered directly to the tumor excision site (e.g., applied by swabbing, brushing or otherwise coating the resection margins of the tumor with the antibody). Alternatively, the antibodies may be incorporated into known surgical pastes prior to administration. Embodiments of the invention contemplate antibodies that may be applied after hepatic resections for malignancy, and after neurosurgical operations.

Within one embodiment of the present invention, antibodies may be administered to the resection margin of a wide variety of tumors, including for example, breast, colon, brain and hepatic tumors. For example, within one embodiment of the invention, an antibody may be administered to the site of a neurological tumor subsequent to excision, such that the formation of new blood vessels at the site is inhibited.

An agent such as an antibody of the present invention may also be administered in combination with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include platelet factor 4; protamine sulphate; sulphated chitin derivatives (prepared from queen crab shells); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cis-hydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3; Chymostatin; Cyclodextrin Tetradecasulfate; Eponemycin; Camptothecin; Fumagillin; Gold Sodium Thiomalate; anticollagenase-serum; alpha2-antiplasmin; Bisantrene; Lobenzarit disodium (N-(2)-carboxyphenyl-4-chloroanthronilic acid disodium or “CCA”; Thalidomide; Angostatic steroid; AGM-1470; carboxynaminolmidazole; and metalloproteinase inhibitors such as BB94.

An agent such as an antibody of the invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, agents of the invention can be used as a marker or detector of a particular immune system disease or disorder.

An agent such as an antibody of the invention may be useful in treating or detecting deficiencies or disorders of hematopoietic cells. For example, an antibody could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDS), Wiskott-Aldrich Disorder, anemia, thrombocytopenia, or hemoglobinuria.

An agent such as an antibody can also be used to modulate hemostatic (the stopping of bleeding) or thrombolytic activity (clot formation). For example, by increasing hemostatic or thrombolytic activity, antibodies could be used to treat blood coagulation disorders (e.g., afibrinogenemia, factor deficiencies), blood platelet disorders (e.g. thrombocytopenia), or wounds resulting from trauma, surgery, or other causes. Alternatively, agents that can decrease hemostatic or thrombolytic activity could be used to inhibit or dissolve clotting, important in the treatment of heart attacks (infarction), strokes, or scarring.

An agent such as an antibody may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of an agent that can inhibit an immune response, particularly the proliferation, differentiation, or chemotaxis of T-cells, may be an effective therapy in preventing autoimmune disorders.

Examples of autoimmune disorders that can be treated or detected by an agent such as an antibody of the invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by agent such as an antibody of the invention. Moreover, an agent can be used to treat anaphylaxis, hypersensitivity to an antigenic molecule, or blood group incompatibility.

An agent such as an antibody may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of an agent such as an antibody that inhibits an immune response, particularly the proliferation, differentiation, or chemotaxis of T-cells, may be an effective therapy in preventing organ rejection or GVHD.

Similarly, an agent such as an antibody of the invention may also be used to modulate inflammation. For example, antibodies may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

An agent such as an antibody of the invention can be used to treat or detect hyperproliferative disorders, including neoplasms. An agent may inhibit the proliferation of the disorder through direct or indirect interactions. Alternatively, an agent may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by an agent such as an antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, brain, nervous (central and peripheral), lymphatic system, pelvic, skin, soft tissue, spleen, prostate, thoracic, and urogenital.

In some embodiments, an agent may be used to treat, prevent or ameliorate breast cancer. In other embodiments, an agent may be used to treat, prevent or ameliorate brain cancer. In some embodiments, an agent may be used to treat, prevent or ameliorate head and/or neck cancer. In other embodiments, an agent may be used to treat, prevent or ameliorate prostate cancer. In other preferred embodiments, an agent may be used to treat, prevent or ameliorate colon cancer. In other embodiments, an agent may be used to treat, prevent or ameliorate Kaposi's sarcoma.

Similarly, other hyperproliferative disorders can also be treated or detected by an agent such as an antibody of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The vaccine of the invention can be used for prophylactic and acute treatment of humans and animals capable of developing kinds of cancer associated with VEGF-D or VEGF-C or both VEGF-D and VEGF-C.

The vaccine permits active immunization against cancerous diseases associated with VEGF-D or VEGF-C or both VEGF-D and VEGF-C. Thus, prophylaxis can be obtained against such cancerous diseases. In addition, the vaccine can be used to treat an existing cancerous disease or to accompany conventional cancer treatments. Application of the vaccine can completely or partly avoid the considerable disadvantages of conventional cancer treatments such as chemo- or radiotherapy.

The biological activity of an agent of the invention can be measured by standard assays known in the art. Examples include ligand binding assays and Scatchard plot analysis; receptor dimerization assays; cellular phosphorylation assays; tyrosine kinase phosphorylation assays; endothelial cell proliferation assays such as BrdU labeling and cell counting experiments; VEGF-D/VEGF-C-dependent cell proliferation assays; and angiogenesis assays. Methods for measuring angiogenesis are standard. Angiogenesis can be assayed by measuring the number of non-branching blood vessel segments (number of segments per unit area), the functional vascular density (total length of perfused blood vessel per unit area), the vessel diameter, the formation of vascular channels, or the vessel volume density (total of calculated blood vessel volume based on length and diameter of each segment per unit area). These assays can be performed using either purified receptor or ligand or both, and can be performed in vitro or in vivo. These assays can also be performed in cells using a genetically introduced or the naturally-occurring ligand or receptor or both. An agent that inhibits the biological activity of VEGF-D and/or VEGF-C will cause a decrease of at least 10%, preferably 20%, 30%, 40%, or 50%, and more preferably 60%, 70%, 80%, 90% or greater decrease in the biological activity of VEGF-D and/or VEGF-C. The inhibition of biological activity can also be measured by the IC₅₀. Preferably, an agent that inhibits the biological activity of VEGF-D and/or VEGF-C will have an IC₅₀ of less than 100 nM, more preferably less than 10 nM and most preferably less than 1 nM.

Peptides of the invention are also suitable for monitoring the obtained immune response in a vaccinated patient. A peptide of the invention can thus be applied both as a vaccine component and as a diagnostic means for monitoring the success of a vaccination.

If the peptide is used as a diagnostic means, it is preferably conjugated to an immunogenic carrier that was not used for the previous vaccination. When monitoring the success of vaccination, this prevents the diagnostic means from reacting to antibodies that were formed against the carrier fraction of the vaccine and therefore do not serve the purpose of prophylaxis or therapy.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

All references, including any patents or patent application, cited in this specification are hereby incorporated by reference. No admission is made that any reference constitutes prior art. It will be clearly understood that, although a number of prior art publications are referred to herein, this reference does not constitute an admission that any of these documents form part of the common general knowledge in the art, in Australia or in any other country.

### EXAMPLES

The invention is now further described in detail by reference to the following examples. The examples are provided for purposes of illustration only, and are not intended to be limiting unless otherwise specified. Thus, the invention encompasses any and all variations which become evident as a result of the teaching provided herein.

### Example 1

### Materials and Methods

Peptides. A series of synthetic 15-mer peptides (peptides 1 to 35, SEQ ID NOs: 4 to 38 in FIG. 4B, which overlap each other by 12 amino acids) were synthesized (Mimotopes Pty Ltd, Melbourne, Australia). The peptides encompassed the VHD of human VEGF-D (SEQ ID NO: 3, FIG. 4A). Peptide 36 (SEQ ID NO: 39) represented an artificial sequence including a FLAG tag as a positive control for a commercial anti-FLAG mAb (M2; Sigma) that was used as control for non-specific binding to peptides 1 to 35 (SEQ ID NOs: 4 to 38). Biotin was included at the N-terminal of each peptide during synthesis, except that peptide 1 (SEQ ID NO: 4, representing the N-terminal of the VHD) has biotin at the C-terminal. The four-amino acid linker “SGSG” was introduced between the N-terminal of each peptide sequence and biotin to provide more flexibility to peptides after immobilization on streptavidin plates, except peptide 1 (SEQ ID NO: 4) that included the C-terminal linker “GSG”.

ELISA. Reacti-Bind streptavidin high-binding-capacity coated plates (Pierce) were coated with 10 pmol of each peptide in PBS, blocked with 5% milk in PBS-0.1% Tween 20, and incubated with 100 μl of VEGF-D neutralizing monoclonal antibody mAb 4A5 (referred to herein as VDnmAb) or M2 at 2 μg/ml for 1 h at room temperature. Bound mAb was detected with goat anti-mouse IgG coupled with HRP. ABTS was used as the substrate, and the absorbance was read at 405 nm in a multiwell plate reader.

Results

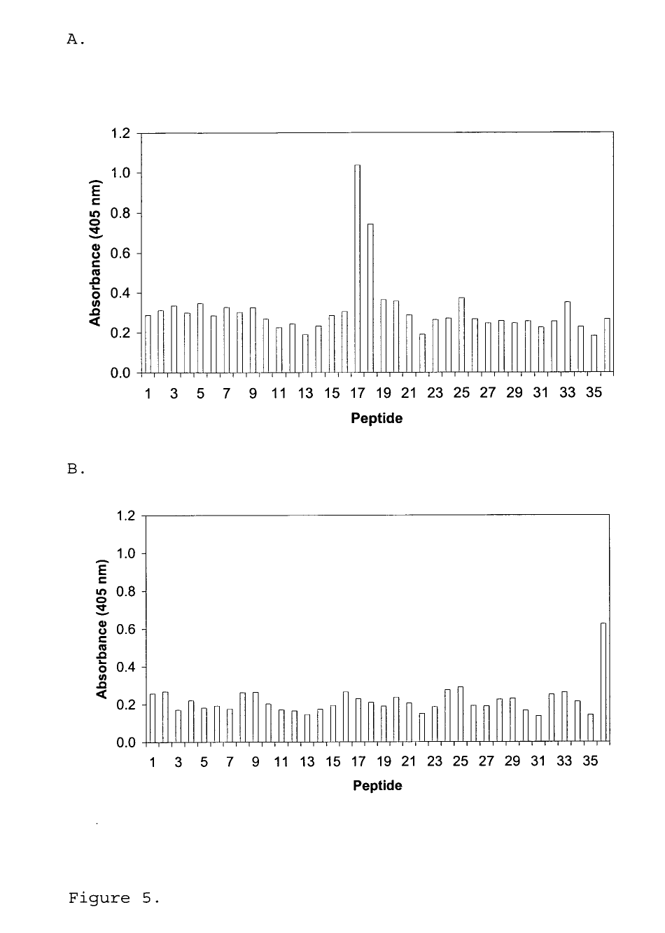

An initial mapping of the VDnmAb binding epitope was carried out with a library of 35 overlapping biotinylated synthetic peptides, spanning the VHD of human VEGF-D (SEQ ID NO: 3, FIG. 4A), by ELISA. Representative results from one of five independent experiments are shown in FIG. 5A. A signal more than two-fold greater than background was considered positive, and anti-FLAG mAb M2 was used as a control for non-specific binding to peptides 1-35 (SEQ ID NOs: 4 to 38; M2 binds specifically to peptide 36, SEQ ID NO: 39 which contains the FLAG sequence) (FIG. 4B). Two positive signals for the interaction with VDnmAb were detected: the strongest with peptide 17 (SEQ ID NO: 20, VNVFRCGGCCNEESL—residues 137 to 151 of human VEGF-D, excluding biotin and linker) and the other with the overlapping peptide 18 (SEQ ID NO: 21, FRCGGCCNEESLICM—residues 140 to 154 of human VEGF-D; excluding biotin and linker). This suggests that the linear epitope recognized by VDnmAb is contained within peptide 17 (SEQ ID NO: 20), and partly within peptide 18 (SEQ ID NO: 21). VDnmAb binds human, but not mouse, VEGF-D. The only residues that differ between human and mouse VEGF-D in the region of peptide 17 (SEQ ID NO: 20) are the two C-terminal residues (“SL” in human, “GV” in mouse). This suggests that the C-terminal region of peptide 17, and the corresponding region of human VEGF-D, may be important for binding VDnmAb. This is consistent with the observations that peptide 16 (SEQ ID NO: 19, which lacks the three C-terminal residues of peptide 17, SEQ ID NO: 20) does not bind VDnmAb, whereas peptide 18 (SEQ ID NO: 21, which contains the C-terminal region of peptide 17, SEQ ID NO: 20) does bind this mAb.

As VDnmAb is a neutralizing monoclonal antibody that blocks binding of human VEGF-D to both VEGFR-2 and VEGFR-3, the epitope to which VDnmAb binds, likely contained within the peptide 17 sequence VNVFRCGGCCNEESL (SEQ ID NO: 20, excluding biotin and linker), may be part of the region of VEGF-D that binds to these receptors. Alternatively, this region might not form part of the receptor-binding surface, but when VDnmAb is bound to this region the antibody might block receptor binding due to steric hindrance.

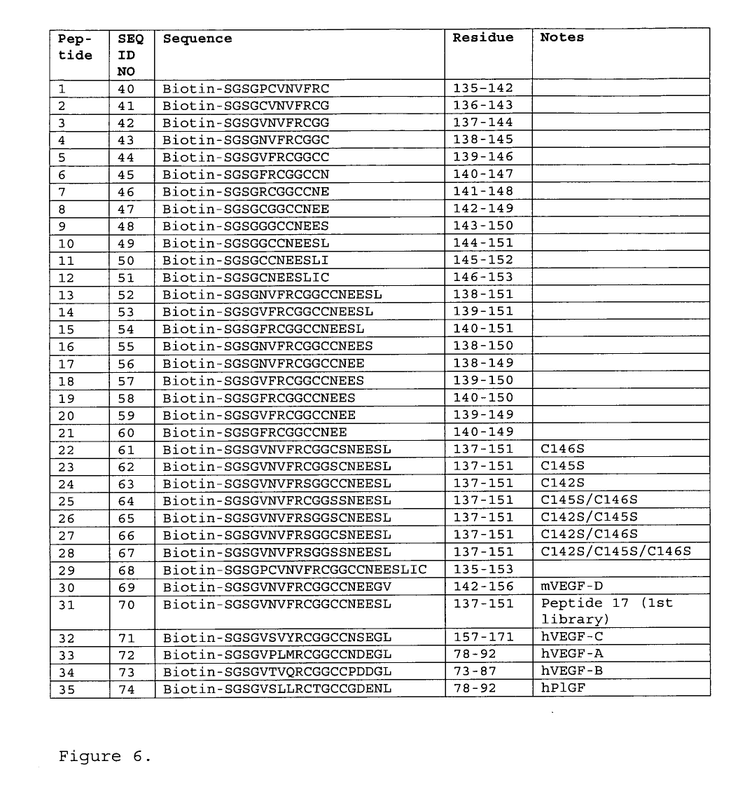

Human VEGF-D and VEGF-C are closely related. The VHDs of these proteins are approximately 60% identical in amino acid sequence, and these growth factors both bind VEGFR-2 and VEGFR-3. Hence, the region of VEGF-C that is homologous to peptide 17 (SEQ ID NO: 20) derived from human VEGF-D may be part of the VEGFR-2 and VEGFR-3 binding sites. This region consists of residues 157 to 171 of human VEGF-C (see peptide 32, SEQ ID NO: 71, of FIG. 6; VSVYRCGGCCNSEGL, excluding biotin and linker). Alternatively, this region of VEGF-C might not form part of the receptor-binding surface, but when a mAb is bound to this site the mAb might block receptor binding due to steric hindrance.

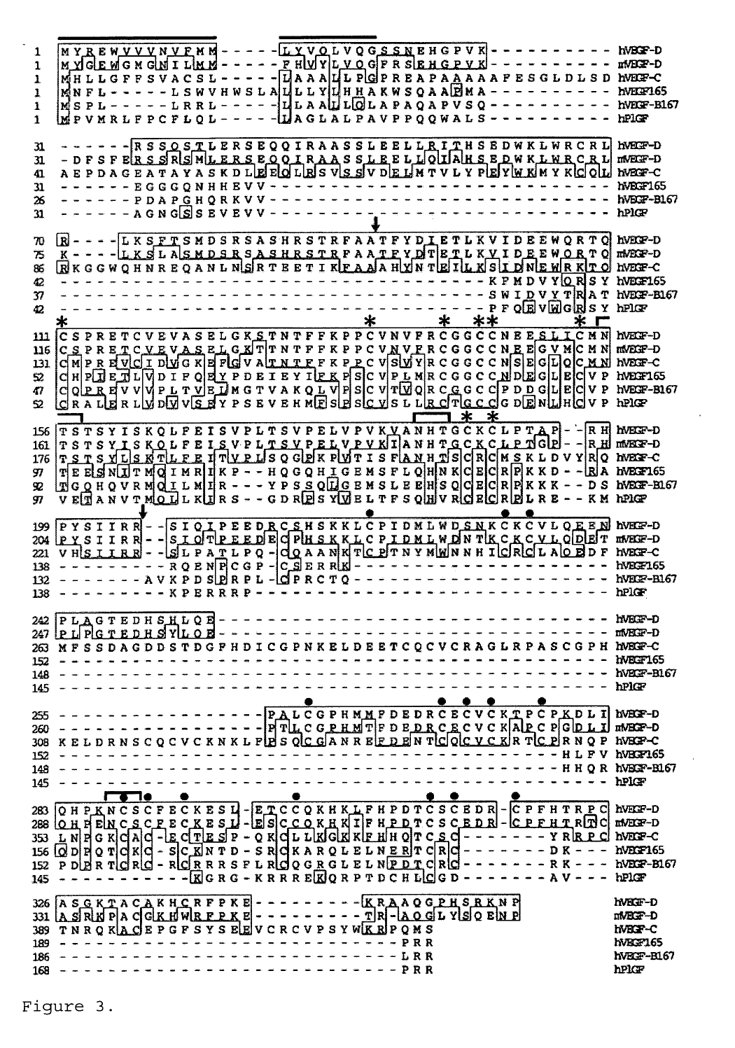

The other members of the human VEGF family are VEGF-A (VEGF), VEGF-B and PlGF. The regions of these growth factors homologous to peptide 17 (SEQ ID NO: 20) derived from human VEGF-D may be important for binding VEGF receptors and/or useful for generating mAbs that interfere with receptor binding. The homologous region consists of residues 78 to 92 of VEGF-A (see peptide 33, SEQ ID NO: 72 of FIG. 6; VPLMRCGGCCNDEGL, excluding biotin and linker); residues 73 to 87 of VEGF-B (see peptide 34, SEQ ID NO: 73 of FIG. 6; VTVQRCGGCCPDDGL, excluding biotin and linker); residues 78 to 92 of PlGF (see peptide 35, SEQ ID NO: 74 of FIG. 6; VSLLRCTGCCGDENL, excluding biotin and linker). All numbering is relative and according to FIG. 1 and FIG. 3 unless stated otherwise.

### Example 2

### Materials and Methods

A series of synthetic peptides (“the 2nd peptide library”), varying in size from 8 to 19 amino acid residues (peptides 1 to 35, SEQ ID NOs: 40 to 74 in FIG. 6) and which included the region of human VEGF-D exhibiting the strongest binding to VDnmAb as assessed by screening of the 1st peptide library, were synthesized (Mimotopes Pty Ltd, Melbourne, Australia). In this 2nd peptide library, biotin was included at the N-terminal of each peptide, and the four-amino acid linker “SGSG” was introduced between each peptide sequence and the biotin residue to provide more flexibility to peptides after immobilization on streptavidin plates. ELISA-based analysis of the 2nd peptide library was carried out as for the 1st peptide library.

Results

Screening of the 1st peptide library (Example 1) revealed that VDnmAb binding epitope may be localized within the sequence VNVFRCGGCCNEESL (peptide 17, SEQ ID NO: 20, excluding biotin and linker), corresponding to residues 137 to 151 of human VEGF-D. To further define the VDnmAb binding epitope, a 2nd peptide library was used consisting of peptides related to peptide 17 (SEQ ID NO: 20)—some were truncated peptides, others contained amino-acid substitutions, others overlapped peptide 17, others represented homologous regions of other VEGF family members, and one peptide (peptide 31, SEQ ID NO: 70) was identical to peptide 17 (SEQ ID NO: 20) of the first library (FIG. 4B). Representative results from one of five independent ELISA experiments analyzing interaction of the peptides with VDnmAb are shown in FIG. 7A; a signal more than two-fold greater than background was considered positive, and anti-FLAG mAb (M2) was used as control for non-specific binding to the peptides (FIG. 7B).

Peptide 31 (SEQ ID NO: 70) of the second library, which contains the region of human VEGF-D present in peptide 17 (SEQ ID NO: 20) from the 1st library, bound to VDnmAb as expected. Three positive signals were detected for shorter variants of peptide 17: the strongest with peptide 14 of the 2nd library (VFRCGGCCNEESL, excluding biotin and linker—corresponding to residues 139 to 151 of human VEGF-D; SEQ ID NO: 53) and two others with peptides 13 (NVFRCGGCCNEESL, excluding biotin and linker—residues 138 to 151 of human VEGF-D; SEQ ID NO: 52) and 15 (FRCGGCCNEESL, excluding biotin and linker—residues 140 to 151 of human VEGF-D; SEQ ID NO: 54). This strongly suggests that the epitope recognized by VDnmAb is contained within residues 139-151 and possibly within residues 140-151. Deletion from peptide 14 (SEQ ID NO: 53) of the leucine residue corresponding to position 151 leads to complete loss of VDnmAb binding (see result for peptide 18 of the 2nd library, SEQ ID NO: 57), indicating the crucial importance of this residue for interaction with VDnmAb. Further, double mutation of serine 150 to glycine, and of leucine 151 to valine (these changes correspond to the sequence of mouse VEGF-D) also leads to complete loss of VDnmAb binding (see result for peptide 30 of the 2nd library, SEQ ID NO: 69).

Peptides representing short overlaps across the VDnmAb binding region (8-mers with 1 amino-acid shift) did not bind the antibody (see results for peptides 1 to 12 of the 2nd library, SEQ ID NOs: 40 to 51). Single mutations of cysteine residues in the binding region, namely C146S or C145S or C142S (peptides 22, 23 and 24 of 2nd library, SEQ ID NOs: 61, 62 and 63) showed very minor or no effect on VDnmAb binding. Mutation of two of the cysteine residues (peptides 25, 26 and 27 of 2nd library, SEQ ID NOs: 64, 65 and 66) had more effect and mutation of all three cysteine residues (peptide 28 of 2nd library, SEQ ID NO: 67) led to significant loss of antibody binding. Peptides corresponding to the regions of VEGF-A, VEGF-B, PlGF and VEGF-C that are homologous to the part of VEGF-D that binds VDnmAb did not bind the antibody (peptide 32, SEQ ID NO: 71, VEGF-C; peptide 33, SEQ ID NO: 72, VEGF-A; peptide 34, SEQ ID NO: 73, VEGF-B; peptide 35, SEQ ID NO: 74, PlGF: 2nd library).

These findings show that the VDnmAb binding epitope in human VEGF-D is contained within residues 139 to 151, and possibly within residues 140 to 151. Further, the leucine residue at position 151 is extremely important for the interaction with the antibody.

### Example 3

### Materials and Methods

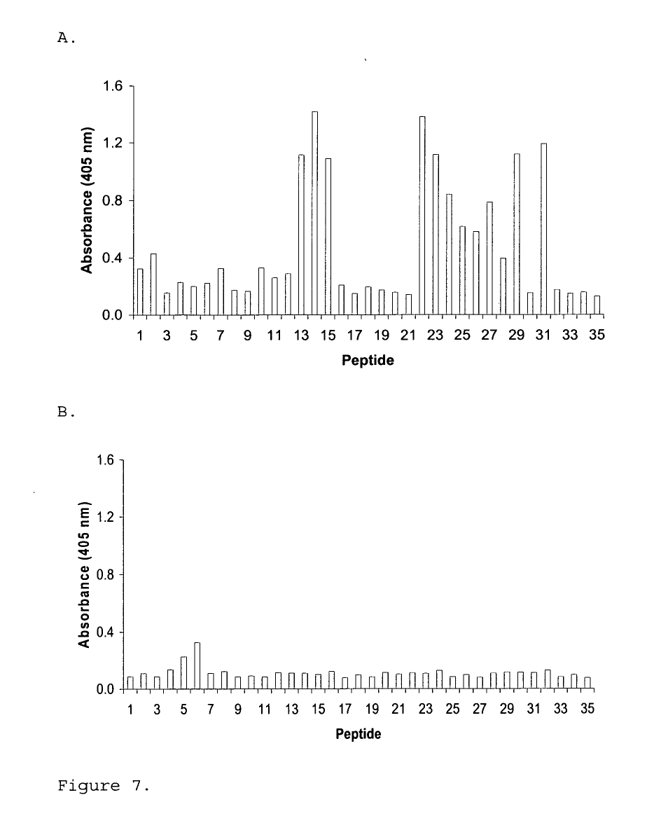

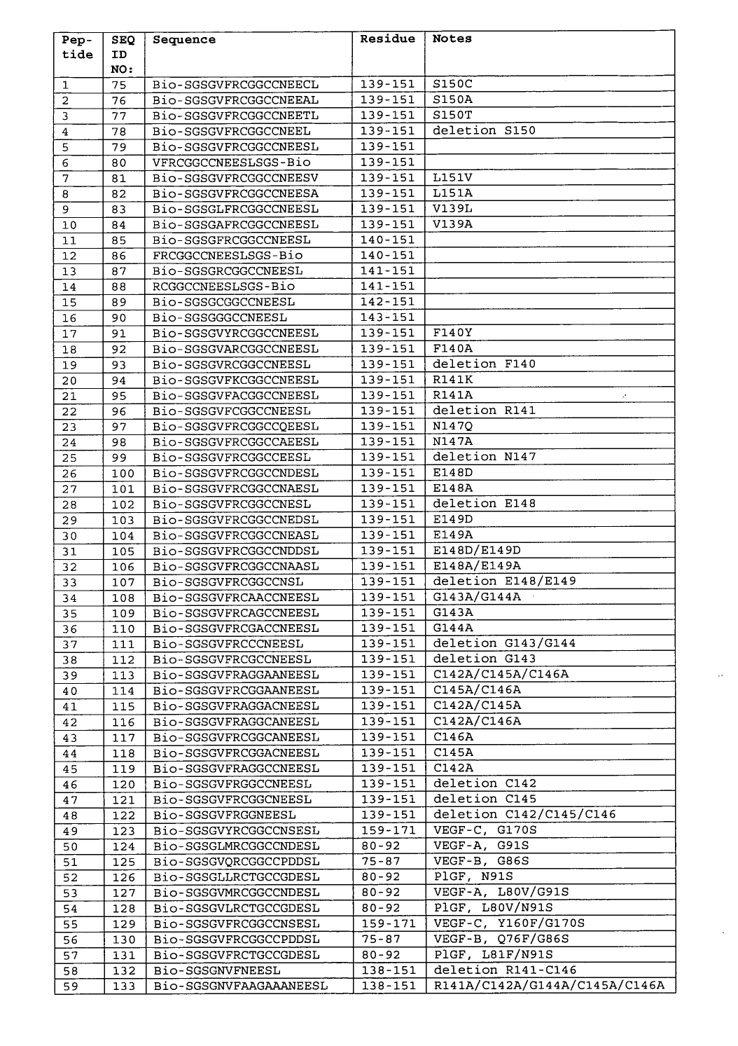

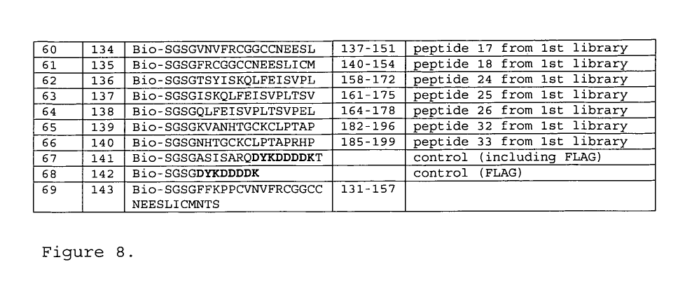

A series of synthetic peptides (“the 3rd peptide library”), varying in size from 8 to 27 amino acid residues (peptides 1 to 69, SEQ ID NOs: 75 to 143 in FIG. 8) and which encompassed the region of human VEGF-D exhibiting the strongest binding to VDnmAb from the first and second peptide libraries, was synthesized (Mimotopes Pty Ltd, Melbourne, Australia). Peptide 67 (SEQ ID NO: 141, the same as peptide 36, SEQ ID NO: 39 from the 1st library) and peptide 68 (SEQ ID NO: 142), both containing the FLAG tag, were used as controls. In this 3rd peptide library, biotin was included at the N-terminal of each peptide except peptides 6, 12 and 14 (SEQ ID NOs: 80, 86 and 88) which have C-terminal biotin residues. To provide more flexibility to peptides after immobilization on streptavidin plates, an amino acid linker was introduced between biotin residues and the peptide sequence: “SGSG” for N-terminal biotin; “SGS” for C-terminal biotin. ELISA-based analysis of the 3rd peptide library was carried out as for the 1st peptide library, described in Example 1.

Results

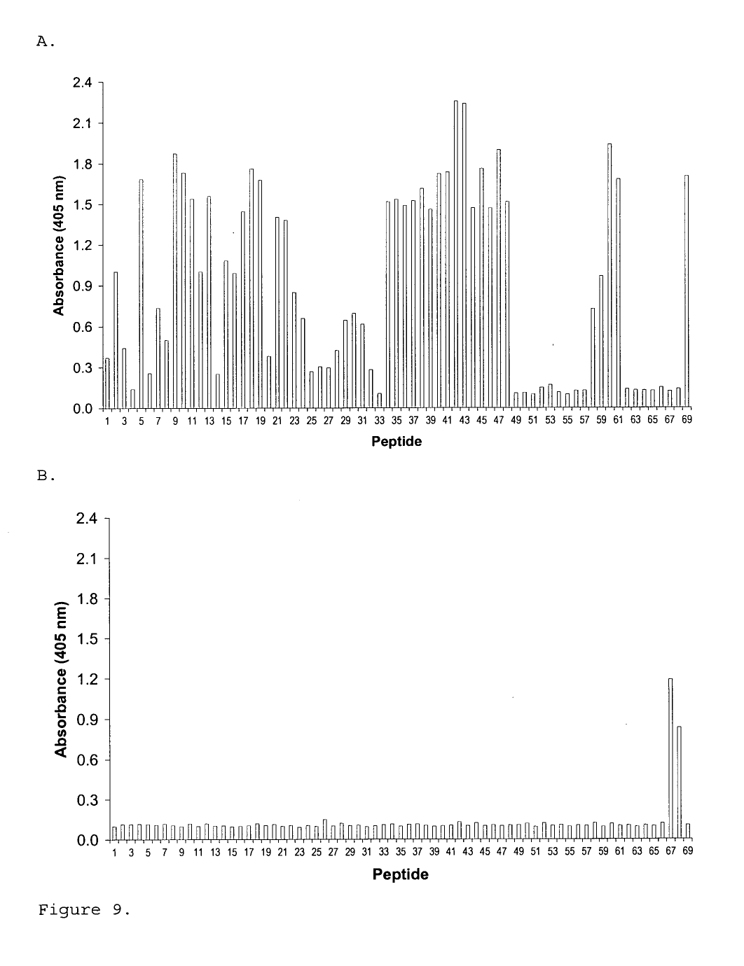

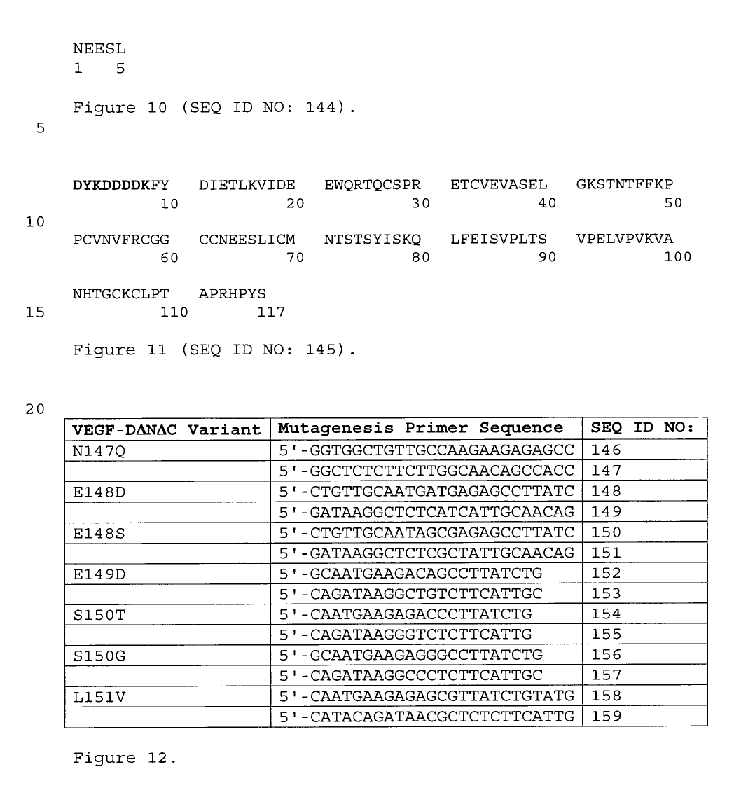

Screening of the 2nd peptide library (Example 2) revealed that the VDnmAb binding epitope is localized within the sequence VFRCGGCCNEESL, corresponding to residues 139 to 151 of human VEGF-D (SEQ ID NO: 1; peptide 14, SEQ ID NO: 53 of the 2nd peptide library also comprised this region). To further define the VDnmAb binding epitope, a 3rd library consisting of peptides related to peptide 14 (SEQ ID NO: 53) of the 2nd peptide library was constructed. Some peptides were truncated variants of peptide 14, having N-terminal or C-terminal biotin residues (peptides 11 to 16, SEQ ID NOs: 85 to 90). Other peptides contained amino acid substitutions or deletions (peptides 1 to 4, 7 to 10, 17 to 48, 58 and 59, SEQ ID NOs: 75 to 78, 81 to 84, 91 to 122, 132 and 133, respectively). Other peptides represented homologous regions of other VEGF family members, but with amino acid substitutions to make these regions more like VEGF-D (peptides 49 to 57, SEQ ID NOs: 123 to 131, FIG. 8). Peptide 5 (SEQ ID NO: 79) was identical to, and peptide 6 (SEQ ID NO: 80) was similar to, peptide 14 (SEQ ID NO: 53) of the 2nd peptide library. The biotin residue of peptide 6 (SEQ ID NO: 80) was at the C-terminal, rather than the N-terminal. Peptides 60, 61, 62, 63, 64, 65 and 66 (SEQ ID NOs: 134 to 140) were identical to peptides 17, 18, 24, 25, 26, 32 and 33 (SEQ ID NOs: 20, 21, 27, 28, 29, 35 and 36, respectively) of the 1st peptide library (FIG. 4B), respectively. Representative results from one of five independent ELISA experiments analyzing the interaction of the peptides with the VEGF-D neutralizing antibody are shown in FIG. 9A; a signal more than two-fold over background was considered positive, and anti-FLAG mAb (M2) was used as control for non-specific binding to the peptides (FIG. 9B).

Peptide 5 (SEQ ID NO: 79) of the third library, which comprises the region of human VEGF-D present in peptide 14 (SEQ ID NO: 53) from the 2nd library, bound to VDnmAb as expected (FIG. 9A). Four positive signals were detected for shorter variants of peptide 5: the strongest with peptide 11 (SEQ ID NO: 85, FRCGGCCNEESL—corresponding to residues 140 to 151 of human VEGF-D, SEQ ID NO: 1) and peptide 13 (SEQ ID NO: 87, RCGGCCNEESL—residues 141 to 151 of SEQ ID NO: 1); and two others with peptide 15 (SEQ ID NO: 89, CGGCCNEESL—residues 142 to 151 of SEQ ID NO: 1) and peptide 16 (SEQ ID NO: 90, GGCCNEESL—residues 143 to 151 of SEQ ID NO: 1). Peptides 6, 12 and 14 (SEQ ID NOs: 80, 86 and 88), which represent the same amino acid sequences as peptides 5, 11 and 13 (SEQ ID NOs: 79, 85 and 87), respectively, but with C-terminal instead of N-terminal biotin residues, showed decreases in VDnmAb binding compared to the N-terminal biotinylated peptides. This could be due to steric hindrance upon immobilization of peptides 6, 12 and 14 on the ELISA plate, which restricts access of VDnmAb to its binding site. Mutations V139L, V139A (peptides 9 and 10, SEQ ID NOs: 83 and 84, respectively), F140Y, F140A (peptides 17 and 18, SEQ ID NOs: 91 and 92, respectively) and R141A (peptide 21, SEQ ID NO: 95), as well as deletions of V139 (peptide 11, SEQ ID NO: 85), F140 (peptide 19, SEQ ID NO: 93) or R141 (peptide 22, SEQ ID NO: 96) had little or no effect on VDnmAb binding. Mutation R141K had a more pronounced effect (peptide 20, SEQ ID NO: 94). Single or double deletions of cysteine and glycine residues at positions 142 to 146, or mutations of these residues to alanine had minor or no effect on VDnmAb binding (peptides 34 to 48, SEQ ID NOs: 108 to 122), while simultaneous changes of most of these residues to alanine (peptide 59, SEQ ID NO: 133) or their deletion (peptide 58, SEQ ID NO: 132) led to loss, but not complete loss, of binding.

Screening of the second peptide library demonstrated the importance of the leucine residue at position 151 for the interaction with VDnmAb. Its deletion led to complete loss of VDnmAb binding (see result for peptide 18, SEQ ID NO: 57 of the 2nd library, FIG. 7A). Screening of the 3rd peptide library showed that mutation of residue 151 of SEQ ID NO: 1 to valine or alanine led to a decreased VDnmAb binding (peptide 7 and peptide 8, SEQ ID NOs: 81 and 82 FIG. 9A). The four amino acids preceding L151 (N147, E148, E149 and S150) are also critical for VDnmAb binding: deletion of any of them led to loss of VDnmAb binding (peptides 4, 25, 28 and 33, SEQ ID NOs: 78, 99, 102 and 107, respectively). Mutation S150A reduced VDnmAb binding somewhat (peptide 2, SEQ ID NO: 76), and mutations S150C or S150T (peptides 1 and 3, SEQ ID NOs: 75 and 77, respectively), N147Q or N147A (peptides 23 and 24, SEQ ID NOs: 97 and 98, respectively), E148D or E148A (peptides 26 and 27, SEQ ID NOs: 100 and 101, respectively), E149D or E149A (peptides 29 and 30, SEQ ID NOs: 103 and 104, respectively), and double mutations E148D/E149D or E148A/E149A (peptides 31 and 32, SEQ ID NOs: 105 and 106, respectively) led to loss, even complete loss, of VDnmAb binding.

In summary, screening of the three peptide libraries revealed that the asparagine residue at position 147, the glutamic acid residues at positions 148 and 149, the serine residue at position 150, and the leucine residue at position 151 of human VEGF-D (SEQ ID NO: 1) are all extremely important for the interaction of VEGF-D with VDnmAb.

### Example 4

In vivo methods can be used to test antibodies of the invention generated using a peptide of the invention as an immunogen. To this end, a xenograft model can be used.

In this model, subcutaneous tumors are established with a cancer cell line by injecting BALB/c athymic nude mice (T-cell deficient) in the right flank with 2×10⁶ tumor cells. Tumors will be allowed to reach 150-200 mm³, and then mice randomized to receive either intraperitoneal injection of anti-VEGF-D or anti-VEGF-C antibodies or non-specific antibodies (control) every 3 days. Subsequently, tumor growth rate will be assessed by monitoring tumor size over time. Subcutaneous tumors will be measured at least weekly using calipers, and tumor volumes will be estimated based on the assumption that tumors are spherical. At the end of the experiment, the animals will be killed. Tumor angiogenesis will be assessed by immunohisto-chemistry for marker PECAM-1. Tumor lymphangiogenesis will be assessed by immunohistochemistry for LYVE-1. Metastatic spread to lymph nodes will be assessed by looking for tumor cells in the lymph nodes. Tumor parameters will be compared between treated and control mice.

### Example 5

Peptides comprising SEQ ID NO: 70 or SEQ ID NO: 71 may be permuted by substitution with an amino acid residue. Initially, SEQ ID NO: 70 may be permuted by substitution of amino acid residues corresponding with positions 147, 148, 149, or 150 of SEQ ID NO: 1. The amino acid residues to be substituted into SEQ ID NO: 70 (hVEGF-D) may be selected from homologous residues derived from SEQ ID NO: 69 or SEQ ID NO: 71. The amino acid residues of SEQ ID NO: 69 or SEQ ID NO: 71 corresponding with positions 147, 148, 149, 150 or 151 of SEQ ID NO: 1 may be responsible for the failure of peptides comprising SEQ ID NO: 69 (mVEGF-D) or SEQ ID NO: 71 (hVEGF-C) from binding to VDnmAb.

By substituting these amino acid residues, those residues critical for peptide binding to VDnmAb may be determined. Alternatively, conservative amino acid residues may be substituted into the peptide comprising SEQ ID NO: 70. Alternatively, non-conservative amino acid residues may be substituted into the peptide comprising SEQ ID NO: 70. Alternatively, amino acid residues may be substituted into the peptide comprising SEQ ID NO: 70 at positions other than those corresponding with positions 147, 148, 149, 150 or 151 of SEQ ID NO: 1.

By substituting an amino acid residue in SEQ ID NO: 70, it is possible to produce a variant peptide that maintains binding to VDnmAb. Consequently, it may be possible to use that permuted peptide to generate an antibody that specifically binds native VEGF-D and native VEGF-C.

### Example 6

Using a technique such as ELISA, it may be possible to determine whether peptides comprising SEQ ID NO: 70 or SEQ ID NO: 71 specifically bind to receptors, including VEGFR-2 and VEGFR-3. Soluble VEGFR-2 (R&D Systems, Inc.) or soluble VEGFR-3 (R&D Systems, Inc.) may be used for this purpose. Alternatively, a cell survival bioassay may be used. Such a bioassay comprises Ba/F3 cells that were stably transfected with a chimeric receptor containing the extracellular domain of human VEGFR-2 or VEGFR-3 and the transmembrane and cytoplasmic domains of the mouse erythropoietin receptor (EpoR) (Stacker et al. 1999 J Biol Chem 274: 34884-34892; Achen et al. 2000 Eur J Biochem 267: 2505-2515).

### Example 7

### Materials and Methods

Site-directed mutagenesis. DNA encoding VEGF-DΔNΔC, a form of mature human VEGF-D tagged at the N-terminus with the FLAG octapeptide (FIG. 11; SEQ ID NO: 145), was subjected to site-directed mutagenesis by PCR to generate VEGF-DΔNΔC variants. Mutations were introduced by amplification with the following primers: N147Q with 5′-GGTGGCTGTTGCCAAGAAGAGAGCC (SEQ ID NO: 146) and 5′-GGCTCTCTTCTTGGCAACAGCCACC (SEQ ID NO: 147); E148D with 5′-CTGTTGCAATGATGAGAGCCTTATC (SEQ ID NO: 148) and 5′-GATAAGGCTCTCATCATTGCAACAG (SEQ ID NO: 149); E148S with 5′-CTGTTGCAATAGCGAGAGCCTTATC (SEQ ID NO: 150) and 5′-GATAAGGCTCTCGCTATTGCAACAG (SEQ ID NO: 151); E149D with 5′-GCAATGAAGACAGCCTTATCTG (SEQ ID NO: 152) and 5′-CAGATAAGGCTGTCTTCATTGC (SEQ ID NO: 153); S150T with 5′-CAATGAAGAGACCCTTATCTG (SEQ ID NO: 154) and 5′-CAGATAAGGGTCTCTTCATTG (SEQ ID NO: 155); S150G with 5′-GCAATGAAGAGGGCCTTATCTG (SEQ ID NO: 156) and 5′-CAGATAAGGCCCTCTTCATTGC (SEQ ID NO: 157); L151V with 5′-CAATGAAGAGAGCGTTATCTGTATG (SEQ ID NO: 158) and 5′-CATACAGATA ACGCTCTCTTCATTG (SEQ ID NO: 159) (FIG. 12). The desired mutations, as well as the absence of any unwanted mutations, in the resulting DNA fragments were confirmed by nucleotide sequencing.

Expression of VEGF-DΔNΔC variants. Plasmids containing DNA encoding VEGF-DΔNΔC or its variants were used for transient transfection of 293-F cells with the FreeStyle MAX 293 Expression System according to manufacturer's instructions (Invitrogen). Cells expressing each variant were cultured in serum-free medium, and 30 ml of conditioned media was collected 7 days post-transfection and used for analysis.

Western Blot Analysis. Conditioned media containing different variants of VEGF-DΔNΔC were resolved by SDS-PAGE, transferred to nitrocellulose membrane, probed with M2 anti-FLAG or VDnmAb antibodies labeled with IRDye 800CW according to manufacturer's instructions (LI-COR), and detected with an Odyssey Infrared Imaging System (LI-COR).

ELISA Analysis. For analysis of VEGF-DΔNΔC variants, microtitre plates (Linbro/Titertek, ICN Biomedicals) were coated with M2 anti-FLAG monoclonal antibody at 5 μg/ml in 100 mM carbonate buffer pH 9.5, then blocked with 1% BSA in PBS-0.1% Tween 20, and incubated with 100 μl of conditioned media containing different variants of VEGF-DΔNΔC for 1 h at room temperature. Bound VEGF-DΔNΔC was detected with anti-VEGF-D MAB286 from R&D Systems (an antibody that binds a different epitope than that bound by VDnmAb; data not shown) as control or VDnmAb coupled with HRP. The assay was developed with an ABTS substrate system (Zymed) and quantified by reading absorbance at 405 nm in a multiwell plate reader.

Results

Screening of the peptide libraries (Examples 1 to 3) suggested that the asparagine residue at position 147, the glutamic acid residues at positions 148 and 149, the serine residue at position 150 and the leucine residue at position 151 of human VEGF-D are important for the interaction with VDnmAb. To validate the importance of these residues for the interaction of VDnmAb with intact VEGF-D protein, variants of VEGF-DΔNΔC were generated using site-directed mutagenesis in which each of these amino acids was altered to a residue closely related in structure; the mutations introduced generated the variants N147Q, E148D, E149D, S150T and L151V.

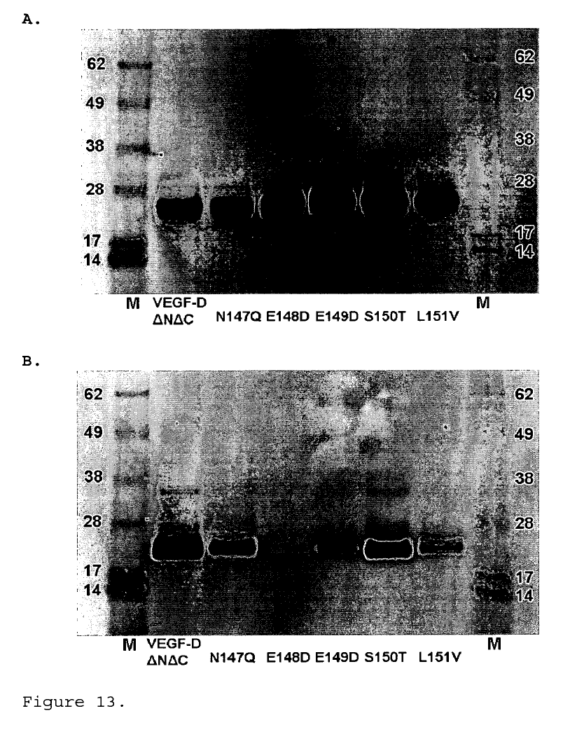

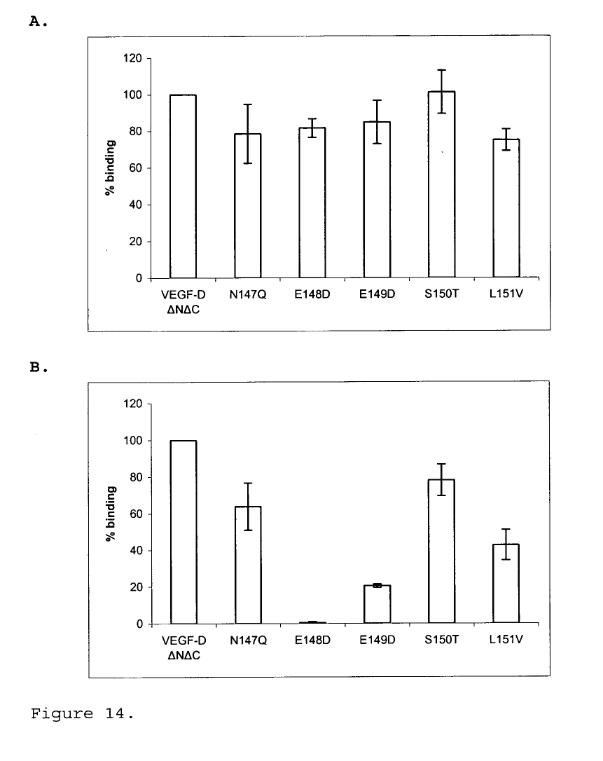

VEGF-DΔNΔC and its variants were expressed in 293-F cells, and the conditioned media containing these proteins were analysed by Western blot and ELISA. In the Western blotting analysis, binding of VDnmAb to all of the variants was reduced in comparison to VEGF-DΔNΔC (FIG. 13) indicating that all the mutations analyzed affected recognition of VEGF-D by VDnmAb. The E148D mutation led to complete loss of VDnmAb binding, E149D and L151V led to a dramatic decrease in the amount of VDnmAb bound and both N147Q and S150T led to a significant decrease in the amount of VDnmAb bound in these blots. Similar results were obtained from ELISA analysis of the binding of VDnmAb by the variants of VEGF-DΔNΔC (FIG. 14). These results are in agreement with those obtained earlier from screening of the peptide libraries. The data indicates that all of the amino acid residues from position 147 to 151 inclusive contribute to the binding of VDnmAb to VEGF-D.

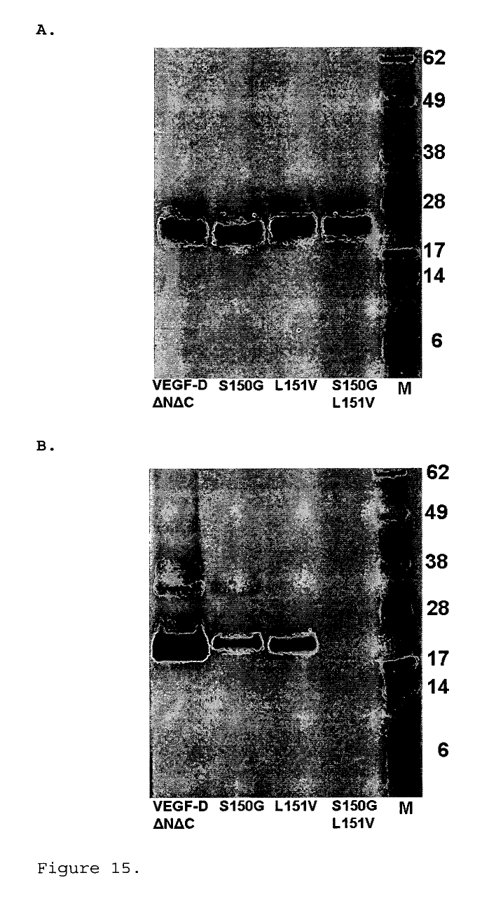

VDnmAb binds human, but not mouse, VEGF-D. The only residues that differ between human and mouse VEGF-D in the region analyzed here (i.e. residues 147-151) are S150 and L151 in human VEGF-D which are G150 and V151 in mouse. Western blotting was used to test conditioned media with human VEGF-DΔNΔC variants bearing a single mutation of each of these residues, and a variant with both mutations (FIG. 15). Both single mutations S150G and L151V significantly affected VDnmAb binding, and the double mutation S150G/L151V led to a complete loss of VDnmAb binding, which is in agreement with the results for peptide 30 of the 2nd peptide library (Example 2).

Human VEGF-D and VEGF-C are closely related in primary structure—the VHDs of these proteins are approximately 60% identical in amino acid sequence. Nevertheless, VDnmAb binds human VEGF-D but does not bind human VEGF-C. The region of five amino acids of VEGF-D analyzed here, which is critical for VDnmAb binding, contains two residues which differ between VEGF-D and VEGF-C—E148 and 5150 of VEGF-D are S and G, respectively, in VEGF-C. In order to understand why VDnmAb does not bind human VEGF-C, VEGF-DΔNΔC variants E148S and S150G and a variant containing both of these mutations were generated. Western blot analysis revealed that the single mutation E148S or the E148S and S150G double mutation resulted in complete loss of VDnmAb binding (data not shown), whereas S150G alone significantly affected, but did not eliminate, binding (FIG. 15). This data indicates that the alteration of E148 in VEGF-D to S in VEGF-C can explain the inability of VDnmAb to bind VEGF-C.

### Example 8

### Materials and Methods

Antibody production. A 15-mer peptide, CAAAANEESLAAAAA (FIG. 16; SEQ ID NO: 160), synthesized by the mild Fmoc chemistry method, was purified by HPLC, conjugated to Keyhole Limpet Hemocyanin (Mimotopes Pty Ltd, Melbourne, Australia) and used to immunize rabbits (Institute of Medical and Veterinary Science, South Australia). Affinity purification of antisera was performed with the immunization peptide coupled to Thiopropyl-Sepharose 6B (Mimotopes Pty Ltd). The affinity-purified antibodies were designated “rabbit NEESL”.

ELISA. For testing rabbit antisera, microtitre plates were coated with VEGF-DΔNΔC at 2 μg/ml in 100 mM carbonate buffer pH 9.5, then blocked with 1% BSA in PBS-0.1% Tween 20, and incubated with serial dilutions of affinity-purified antiserum in blocking solution. Rabbit antibodies bound to VEGF-DΔNΔC were detected with HRP-conjugated secondary anti-rabbit antibodies (Zymed). The assay was developed with an ABTS substrate system (Zymed) and quantified by reading absorbance at 405 nm in a multiwell plate reader.

Bioassays for Binding and Cross-Linking the Extracellular Domains of VEGFR-2 or VEGFR-3. These bioassays employed cell lines expressing chimeric receptors consisting of the extracellular domain of mouse VEGFR-2 or human VEGFR-3 and the transmembrane and cytoplasmic domains of the mouse erythropoietin receptor, designated VEGFR-2-EpoR-Ba/F3 or VEGFR-3-EpoR-Ba/F3 cells. Binding and cross-linking of the VEGFR-2 or VEGFR-3 chimeric receptors allows these cells to survive and proliferate in the absence of interleukin-3 (IL-3). Samples of purified human VEGF-D (consisting of the central VHD of the protein; R&D Systems) at 0.25 μg/ml were incubated with affinity-purified rabbit antibodies or VDnmAb at 25 μg/ml for 30 min at 4° C. The mixtures were added to VEGFR-2-EpoR-Ba/F3 and VEGFR-3-EpoR-Ba/F3 cells in 96-well plates, followed by incubation for 48 h at 37° C. in the absence of IL-3. DNA synthesis was then quantified by the addition of 1 μCi of ³H-thymidine and further incubation for 4 h prior to harvesting using an automated cell harvester (Tomtec, Packard, USA). Incorporated ³H-thymidine was measured by β-counting (Can berra Packard “Top Count NXT™” scintillation counter, USA). Incubation of all antibodies used in the bioassays with VEGFR-2-EpoR-Ba/F3 or VEGFR-3-EpoR-Ba/F3 cells in the absence of VEGF-D, but in the presence of IL-3, revealed that the antibodies were not toxic to the cell lines at the concentration used in this example (i.e. 25 μg/ml).

Results

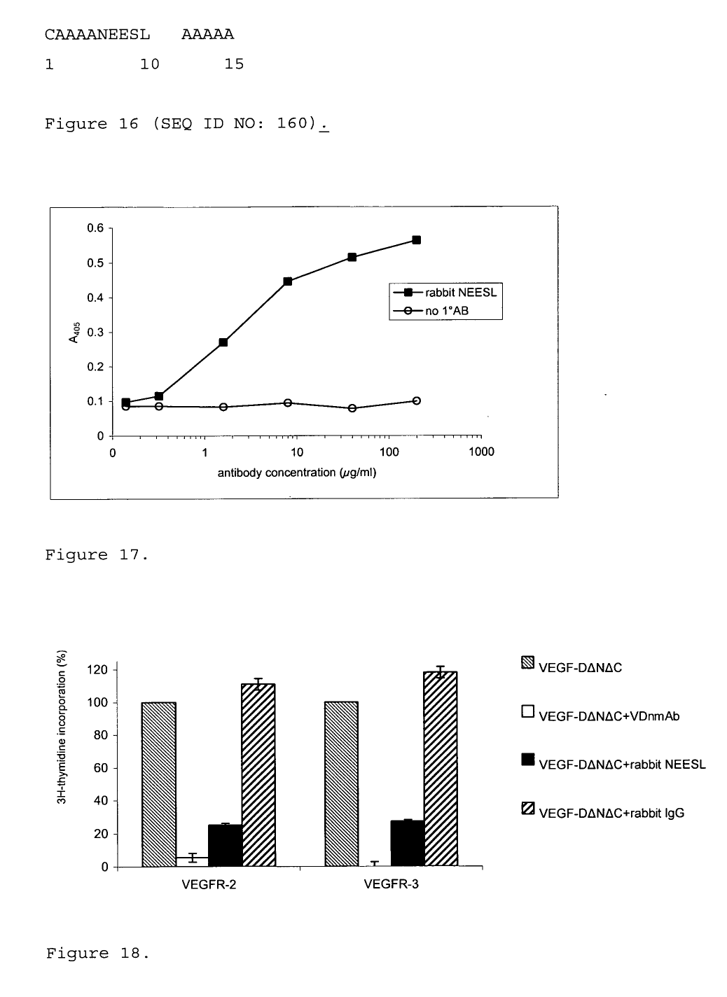

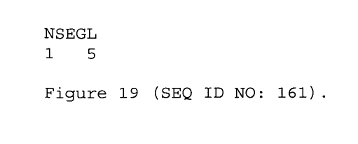

To test the possibility of using a peptide containing the five amino acid residues NEESL (positions 147 to 151 of VEGF-D, that are involved in the binding of VDnmAb) for generating neutralizing antibodies to VEGF-D, affinity-purified rabbit polyclonal antibodies were generated to the peptide CAAAANEESLAAAAA (SEQ ID NO: 160; the alanine residues surrounding the NEESL motif in this peptide all differ from the residues surrounding this motif in human VEGF-D). Analysis of the affinity-purified antibodies (designated “rabbit NEESL”) by ELISA demonstrated VEGF-D-binding activity (FIG. 17). The capacity of rabbit NEESL to block the binding and cross-linking of the extracellular domains of VEGFR-2 and VEGFR-3 by a mature form of human VEGF-D was assessed in cell-based bioassays. In both the VEGFR-2 and VEGFR-3 bioassays, rabbit NEESL inhibited the survival and proliferation of cells promoted by VEGF-D (FIG. 18), indicating that the antibodies can bind VEGF-D so as to block binding or cross-linking of the VEGFR-2 and VEGFR-3 extracellular domains. This data shows that a peptide containing the NEESL motif can be used to generate an immune response involving neutralizing VEGF-D antibodies.

1-20. (canceled)

21. An isolated peptide of 5 to 100 amino acids,
wherein the amino acid sequence of the peptide is at least 60% identical over its length to the human VEGF-D amino acid sequence of SEQ ID NO: 1,
wherein 1 or 2 of the residues of the peptide corresponding to positions selected from 147, 148, 149, 150 and 151 of SEQ ID NO: 1 is modified (“the modification”) relative to SEQ ID NO: 1, and
wherein at least one of the 1 or 2 modifications is not N147del, E148del, E149del, S150del, L151del, N147Q, E148A, E148D, E149D, S150C, S150T, S150G or L151V.

22. The isolated peptide of claim 21, wherein the amino acid sequence of the peptide is at least 90% identical over its length to the human VEGF-D amino acid sequence of SEQ ID NO: 1.

23. The isolated peptide according to claim 22, wherein the peptide is 5-30 amino acids.

24. A nucleic acid molecule comprising a nucleotide sequence that encodes a peptide of 5 to 100 amino acids,
wherein the amino acid sequence of the peptide is at least 60% identical over its length to the human VEGF-D amino acid sequence of SEQ ID NO: 1,
wherein 1 or 2 of the residues of the peptide corresponding to positions selected from 147, 148, 149, 150 and 151 of SEQ ID NO: 1 is modified (“the modification”) relative to SEQ ID NO: 1, and
wherein at least one of the 1 or 2 modifications is not N147del, E148del, E149del, S150del, L151del, N147Q, E148A, E148D, E149D, S150C, S150T, S150G or L151V.

25. An antibody that is raised against a peptide of 5 to 100 amino acids,
wherein the antibody binds to the VEGF homology domain (VHD) of VEGF-D (SEQ ID NO: 1) or VEGF-C (SEQ ID NO: 2); and
wherein the amino acid sequence of the peptide is at least 60% identical over its length to the human VEGF-D amino acid sequence of SEQ ID NO: 1,
wherein 1 or 2 of the residues of the peptide corresponding to positions selected from 147, 148, 149, 150 and 151 of SEQ ID NO: 1 is modified (“the modification”) relative to SEQ ID NO: 1, and
wherein at least one of the 1 or 2 modifications is not N147del, E148del, E149del, S150del, L151del, N147Q, E148A, E148D, E149D, S150C, S150T, S150G or L151V.

26. The antibody of claim 25, wherein the antibody is capable of binding to the VEGF homology domains (VHD) of VEGF-D (SEQ ID NO: 1) and VEGF-C (SEQ ID NO: 2).

27. An antibody that is raised against a peptide of 5 to 100 amino acids,
wherein the amino acid sequence of the peptide comprises residues 147-151 of SEQ ID NO: 1 (NEESL), and
wherein the antibody is capable of binding to the VEGF homology domains (VHD) of VEGF-D (SEQ ID NO: 1) and VEGF-C (SEQ ID NO: 2).

28. A method of making an antibody comprising immunizing an animal with a peptide according to claim 21 to raise antibodies, and selecting an antibody that binds the peptide.

29. The method of claim 28, further comprising selecting an antibody that binds to the VEGF homology domain (VHD) of VEGF-D (SEQ ID NO: 1) or VEGF-C (SEQ ID NO: 2).

30. A method of making an antibody comprising immunizing an animal with a peptide according to claim 21 to raise antibodies, and selecting an antibody that binds to the VEGF homology domain (VHD) of VEGF-D (SEQ ID NO: 1) or VEGF-C (SEQ ID NO: 2).

31. A method of making an antibody comprising:
immunizing an animal with a peptide of 5 to 100 amino acids to raise antibodies, wherein the amino acid sequence of the peptide comprises residues 147-151 of SEQ ID NO: 1 (NEESL), and
selecting an antibody that is capable of binding to the VEGF homology domains (VHD) of VEGF-D (SEQ ID NO: 1) and VEGF-C (SEQ ID NO: 2).

32. An immunogenic composition comprising:
(a) the peptide of claim 21; or
(b) a nucleic acid molecule encoding the peptide of (a).

33. A method for treating a condition responsive to neutralizing VEGF-D, VEGF-C, or both VEGF-D and VEGF-C, the method comprising administering to a subject in need of treatment for the condition a therapeutically effective amount of a composition comprising:
(a) the peptide of claim 21;
(b) a nucleic acid molecule encoding the peptide of (a); or
(c) an antibody that is raised against the peptide of (a) and binds to the VEGF homology domain (VHD) of VEGF-D (SEQ ID NO: 1) or VEGF-C (SEQ ID NO: 2).

34. The method according to claim 33, wherein the condition comprises dysregulated angiogenesis, dysregulated lymphangiogenesis, cancer, rheumatoid arthritis, psoriasis, lymphangiolieomyomatosis, or other inflammatory conditions.

35. A method for treating a condition responsive to neutralizing VEGF-D, VEGF-C, or both VEGF-D and VEGF-C, the method comprising administering to a subject in need of treatment for the condition a therapeutically effective amount of a composition comprising the antibody according to claim 27.

36. The method according to claim 35, wherein the condition comprises dysregulated angiogenesis, dysregulated lymphangiogenesis, cancer, rheumatoid arthritis, psoriasis, lymphangiolieomyomatosis, or other inflammatory conditions.

37. A method for determining an amino acid residue of VEGF-D or VEGF-C or a receptor, required for specific binding of VEGF-D or VEGF-C to the receptor, comprising:
(a) contacting VEGF-D or VEGF-C to a receptor in the presence and absence of:
(i) the peptide of claim 21; or
(ii) an antibody that is raised against the peptide of (i) and binds to the VEGF homology domain (VHD) of VEGF-D (SEQ ID NO: 1) or VEGF-C (SEQ ID NO: 2); and
(b) determining an amino acid residue of VEGF-D or VEGF-C required for the specific binding from differential binding between the VEGF-D or VEGF-C and the receptor in the presence versus the absence of the peptide or antibody.

38. The method of claim 37, wherein the receptor is VEGFR-2 or VEGFR-3.

39. The method of claim 37, wherein the receptor is NRP-1, NRP-2, or is an integrin.

40. The method of claim 39, wherein the receptor is integrin α9β1.


SEQUENCE LISTING (161 sequences)

SEQ ID NO: 1 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - protein, 117 aa, Homo sapiens
FAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 4 - protein, 18 aa, Artificial
  Synthetic
FAATFYDIETLKVIDGSG

SEQ ID NO: 5 - protein, 19 aa, Artificial
  Synthetic
SGSGTFYDIETLKVIDEEW

SEQ ID NO: 6 - protein, 19 aa, Artificial
  Synthetic
SGSGDIETLKVIDEEWQRT

SEQ ID NO: 7 - protein, 19 aa, Artificial
  Synthetic
SGSGTLKVIDEEWQRTQCS

SEQ ID NO: 8 - protein, 19 aa, Artificial
  Synthetic
SGSGVIDEEWQRTQCSPRE

SEQ ID NO: 9 - protein, 19 aa, Artificial
  Synthetic
SGSGEEWQRTQCSPRETCV

SEQ ID NO: 10 - protein, 19 aa, Artificial
  Synthetic
SGSGQRTQCSPRETCVEVA

SEQ ID NO: 11 - protein, 19 aa, Artificial
  Synthetic
SGSGQCSPRETCVEVASEL

SEQ ID NO: 12 - protein, 19 aa, Artificial
  Synthetic
SGSGPRETCVEVASELGKS

SEQ ID NO: 13 - protein, 19 aa, Artificial
  Synthetic
SGSGTCVEVASELGKSTNT

SEQ ID NO: 14 - protein, 19 aa, Artificial
  Synthetic
SGSGEVASELGKSTNTFFK

SEQ ID NO: 15 - protein, 19 aa, Artificial
  Synthetic
SGSGSELGKSTNTFFKPPC

SEQ ID NO: 16 - protein, 19 aa, Artificial
  Synthetic
SGSGGKSTNTFFKPPCVNV

SEQ ID NO: 17 - protein, 19 aa, Artificial
  Synthetic
SGSGTNTFFKPPCVNVFRC

SEQ ID NO: 18 - protein, 19 aa, Artificial
  Synthetic
SGSGFFKPPCVNVFRCGGC

SEQ ID NO: 19 - protein, 19 aa, Artificial
  Synthetic
SGSGPPCVNVFRCGGCCNE

SEQ ID NO: 20 - protein, 19 aa, Artificial
  Synthetic
SGSGVNVFRCGGCCNEESL

SEQ ID NO: 21 - protein, 19 aa, Artificial
  Synthetic
SGSGFRCGGCCNEESLICM

SEQ ID NO: 22 - protein, 19 aa, Artificial
  Synthetic
SGSGGGCCNEESLICMNTS

SEQ ID NO: 23 - protein, 19 aa, Artificial
  Synthetic
SGSGCNEESLICMNTSTSY

SEQ ID NO: 24 - protein, 19 aa, Artificial
  Synthetic
SGSGESLICMNTSTSYISK

SEQ ID NO: 25 - protein, 19 aa, Artificial
  Synthetic
SGSGICMNTSTSYISKQLF

SEQ ID NO: 26 - protein, 19 aa, Artificial
  Synthetic
SGSGNTSTSYISKQLFEIS

SEQ ID NO: 27 - protein, 19 aa, Artificial
  Synthetic
SGSGTSYISKQLFEISVPL

SEQ ID NO: 28 - protein, 19 aa, Artificial
  Synthetic
SGSGISKQLFEISVPLTSV

SEQ ID NO: 29 - protein, 19 aa, Artificial
  Synthetic
SGSGQLFEISVPLTSVPEL

SEQ ID NO: 30 - protein, 19 aa, Artificial
  Synthetic
SGSGEISVPLTSVPELVPV

SEQ ID NO: 31 - protein, 19 aa, Artificial
  Synthetic
SGSGVPLTSVPELVPVKVA

SEQ ID NO: 32 - protein, 19 aa, Artificial
  Synthetic
SGSGTSVPELVPVKVANHT

SEQ ID NO: 33 - protein, 19 aa, Artificial
  Synthetic
SGSGPELVPVKVANHTGCK

SEQ ID NO: 34 - protein, 19 aa, Artificial
  Synthetic
SGSGVPVKVANHTGCKCLP

SEQ ID NO: 35 - protein, 19 aa, Artificial
  Synthetic
SGSGKVANHTGCKCLPTAP

SEQ ID NO: 36 - protein, 19 aa, Artificial
  Synthetic
SGSGNHTGCKCLPTAPRHP

SEQ ID NO: 37 - protein, 19 aa, Artificial
  Synthetic
SGSGGCKCLPTAPRHPYSI

SEQ ID NO: 38 - protein, 19 aa, Artificial
  Synthetic
SGSGCLPTAPRHPYSIIRR

SEQ ID NO: 39 - protein, 20 aa, Artificial
  Synthetic
SGSGASISARQDYKDDDDKT

SEQ ID NO: 40 - protein, 12 aa, Artificial
  Synthetic
SGSGPCVNVFRC

SEQ ID NO: 41 - protein, 12 aa, Artificial
  Synthetic
SGSGCVNVFRCG

SEQ ID NO: 42 - protein, 12 aa, Artificial
  Synthetic
SGSGVNVFRCGG

SEQ ID NO: 43 - protein, 12 aa, Artificial
  Synthetic
SGSGNVFRCGGC

SEQ ID NO: 44 - protein, 12 aa, Artificial
  Synthetic
SGSGVFRCGGCC

SEQ ID NO: 45 - protein, 12 aa, Artificial
  Synthetic
SGSGFRCGGCCN

SEQ ID NO: 46 - protein, 12 aa, Artificial
  Synthetic
SGSGRCGGCCNE

SEQ ID NO: 47 - protein, 12 aa, Artificial
  Synthetic
SGSGCGGCCNEE

SEQ ID NO: 48 - protein, 12 aa, Artificial
  Synthetic
SGSGGGCCNEES

SEQ ID NO: 49 - protein, 12 aa, Artificial
  Synthetic
SGSGGCCNEESL

SEQ ID NO: 50 - protein, 12 aa, Artificial
  Synthetic
SGSGCCNEESLI

SEQ ID NO: 51 - protein, 12 aa, Artificial
  Synthetic
SGSGCNEESLIC

SEQ ID NO: 52 - protein, 18 aa, Artificial
  Synthetic
SGSGNVFRCGGCCNEESL

SEQ ID NO: 53 - protein, 17 aa, Artificial
  Synthetic
SGSGVFRCGGCCNEESL

SEQ ID NO: 54 - protein, 16 aa, Artificial
  Synthetic
SGSGFRCGGCCNEESL

SEQ ID NO: 55 - protein, 17 aa, Artificial
  Synthetic
SGSGNVFRCGGCCNEES

SEQ ID NO: 56 - protein, 16 aa, Artificial
  Synthetic
SGSGNVFRCGGCCNEE

SEQ ID NO: 57 - protein, 16 aa, Artificial
  Synthetic
SGSGVFRCGGCCNEES

SEQ ID NO: 58 - protein, 15 aa, Artificial
  Synthetic
SGSGFRCGGCCNEES

SEQ ID NO: 59 - protein, 15 aa, Artificial
  Synthetic
SGSGVFRCGGCCNEE

SEQ ID NO: 60 - protein, 14 aa, Artificial
  Synthetic
SGSGFRCGGCCNEE

SEQ ID NO: 61 - protein, 19 aa, Artificial
  Synthetic
SGSGVNVFRCGGCSNEESL

SEQ ID NO: 62 - protein, 19 aa, Artificial
  Synthetic
SGSGVNVFRCGGSCNEESL

SEQ ID NO: 63 - protein, 19 aa, Artificial
  Synthetic
SGSGVNVFRSGGCCNEESL

SEQ ID NO: 64 - protein, 19 aa, Artificial
  Synthetic
SGSGVNVFRCGGSSNEESL

SEQ ID NO: 65 - protein, 19 aa, Artificial
  Synthetic
SGSGVNVFRSGGSCNEESL

SEQ ID NO: 66 - protein, 19 aa, Artificial
  Synthetic
SGSGVNVFRSGGCSNEESL

SEQ ID NO: 67 - protein, 19 aa, Artificial
  Synthetic
SGSGVNVFRSGGSSNEESL

SEQ ID NO: 68 - protein, 23 aa, Artificial
  Synthetic
SGSGPCVNVFRCGGCCNEESLIC

SEQ ID NO: 69 - protein, 19 aa, Artificial
  Synthetic
SGSGVNVFRCGGCCNEEGV

SEQ ID NO: 70 - protein, 19 aa, Artificial
  Synthetic
SGSGVNVFRCGGCCNEESL

SEQ ID NO: 71 - protein, 19 aa, Artificial
  Synthetic
SGSGVSVYRCGGCCNSEGL

SEQ ID NO: 72 - protein, 19 aa, Artificial
  Synthetic
SGSGVPLMRCGGCCNDEGL

SEQ ID NO: 73 - protein, 19 aa, Artificial
  Synthetic
SGSGVTVQRCGGCCPDDGL

SEQ ID NO: 74 - protein, 19 aa, Artificial
  Synthetic
SGSGVSLLRCTGCCGDENL

SEQ ID NO: 75 - protein, 17 aa, Artificial
  Synthetic
SGSGVFRCGGCCNEECL

SEQ ID NO: 76 - protein, 17 aa, Artificial
  Synthetic
SGSGVFRCGGCCNEEAL

SEQ ID NO: 77 - protein, 17 aa, Artificial
  Synthetic
SGSGVFRCGGCCNEETL

SEQ ID NO: 78 - protein, 16 aa, Artificial
  Synthetic
SGSGVFRCGGCCNEEL

SEQ ID NO: 79 - protein, 17 aa, Artificial
  Synthetic
SGSGVFRCGGCCNEESL

SEQ ID NO: 80 - protein, 16 aa, Artificial
  Synthetic
VFRCGGCCNEESLSGS

SEQ ID NO: 81 - protein, 17 aa, Artificial
  Synthetic
SGSGVFRCGGCCNEESV

SEQ ID NO: 82 - protein, 17 aa, Artificial
  Synthetic
SGSGVFRCGGCCNEESA

SEQ ID NO: 83 - protein, 17 aa, Artificial
  Synthetic
SGSGLFRCGGCCNEESL

SEQ ID NO: 84 - protein, 17 aa, Artificial
  Synthetic
SGSGAFRCGGCCNEESL

SEQ ID NO: 85 - protein, 16 aa, Artificial
  Synthetic
SGSGFRCGGCCNEESL

SEQ ID NO: 86 - protein, 15 aa, Artificial
  Synthetic
FRCGGCCNEESLSGS

SEQ ID NO: 87 - protein, 15 aa, Artificial
  Synthetic
SGSGRCGGCCNEESL

SEQ ID NO: 88 - protein, 14 aa, Artificial
  Synthetic
RCGGCCNEESLSGS

SEQ ID NO: 89 - protein, 14 aa, Artificial
  Synthetic
SGSGCGGCCNEESL

SEQ ID NO: 90 - protein, 13 aa, Artificial
  Synthetic
SGSGGGCCNEESL

SEQ ID NO: 91 - protein, 17 aa, Artificial
  Synthetic
SGSGVYRCGGCCNEESL

SEQ ID NO: 92 - protein, 17 aa, Artificial
  Synthetic
SGSGVARCGGCCNEESL

SEQ ID NO: 93 - protein, 16 aa, Artificial
  Synthetic
SGSGVRCGGCCNEESL

SEQ ID NO: 94 - protein, 17 aa, Artificial
  Synthetic
SGSGVFKCGGCCNEESL

SEQ ID NO: 95 - protein, 17 aa, Artificial
  Synthetic
SGSGVFACGGCCNEESL

SEQ ID NO: 96 - protein, 16 aa, Artificial
  Synthetic
SGSGVFCGGCCNEESL

SEQ ID NO: 97 - protein, 17 aa, Artificial
  Synthetic
SGSGVFRCGGCCQEESL

SEQ ID NO: 98 - protein, 17 aa, Artificial
  Synthetic
SGSGVFRCGGCCAEESL

SEQ ID NO: 99 - protein, 16 aa, Artificial
  Synthetic
SGSGVFRCGGCCEESL

SEQ ID NO: 100 - protein, 17 aa, Artificial
  Synthetic
SGSGVFRCGGCCNDESL

SEQ ID NO: 101 - protein, 17 aa, Artificial
  Synthetic
SGSGVFRCGGCCNAESL

SEQ ID NO: 102 - protein, 16 aa, Artificial
  Synthetic
SGSGVFRCGGCCNESL

SEQ ID NO: 103 - protein, 17 aa, Artificial
  Synthetic
SGSGVFRCGGCCNEDSL

SEQ ID NO: 104 - protein, 17 aa, Artificial
  Synthetic
SGSGVFRCGGCCNEASL

SEQ ID NO: 105 - protein, 17 aa, Artificial
  Synthetic
SGSGVFRCGGCCNDDSL

SEQ ID NO: 106 - protein, 17 aa, Artificial
  Synthetic
SGSGVFRCGGCCNAASL

SEQ ID NO: 107 - protein, 15 aa, Artificial
  Synthetic
SGSGVFRCGGCCNSL

SEQ ID NO: 108 - protein, 17 aa, Artificial
  Synthetic
SGSGVFRCAACCNEESL

SEQ ID NO: 109 - protein, 17 aa, Artificial
  Synthetic
SGSGVFRCAGCCNEESL

SEQ ID NO: 110 - protein, 17 aa, Artificial
  Synthetic
SGSGVFRCGACCNEESL

SEQ ID NO: 111 - protein, 15 aa, Artificial
  Synthetic
SGSGVFRCCCNEESL

SEQ ID NO: 112 - protein, 16 aa, Artificial
  Synthetic
SGSGVFRCGCCNEESL

SEQ ID NO: 113 - protein, 17 aa, Artificial
  Synthetic
SGSGVFRAGGAANEESL

SEQ ID NO: 114 - protein, 17 aa, Artificial
  Synthetic
SGSGVFRCGGAANEESL

SEQ ID NO: 115 - protein, 17 aa, Artificial
  Synthetic
SGSGVFRAGGACNEESL

SEQ ID NO: 116 - protein, 17 aa, Artificial
  Synthetic
SGSGVFRAGGCANEESL

SEQ ID NO: 117 - protein, 17 aa, Artificial
  Synthetic
SGSGVFRCGGCANEESL

SEQ ID NO: 118 - protein, 17 aa, Artificial
  Synthetic
SGSGVFRCGGACNEESL

SEQ ID NO: 119 - protein, 17 aa, Artificial
  Synthetic
SGSGVFRAGGCCNEESL

SEQ ID NO: 120 - protein, 16 aa, Artificial
  Synthetic
SGSGVFRGGCCNEESL

SEQ ID NO: 121 - protein, 16 aa, Artificial
  Synthetic
SGSGVFRCGGCNEESL

SEQ ID NO: 122 - protein, 14 aa, Artificial
  Synthetic
SGSGVFRGGNEESL

SEQ ID NO: 123 - protein, 17 aa, Artificial
  Synthetic
SGSGVYRCGGCCNSESL

SEQ ID NO: 124 - protein, 17 aa, Artificial
  Synthetic
SGSGLMRCGGCCNDESL

SEQ ID NO: 125 - protein, 17 aa, Artificial
  Synthetic
SGSGVQRCGGCCPDDSL

SEQ ID NO: 126 - protein, 17 aa, Artificial
  Synthetic
SGSGLLRCTGCCGDESL

SEQ ID NO: 127 - protein, 17 aa, Artificial
  Synthetic
SGSGVMRCGGCCNDESL

SEQ ID NO: 128 - protein, 17 aa, Artificial
  Synthetic
SGSGVLRCTGCCGDESL

SEQ ID NO: 129 - protein, 17 aa, Artificial
  Synthetic
SGSGVFRCGGCCNSESL

SEQ ID NO: 130 - protein, 17 aa, Artificial
  Synthetic
SGSGVFRCGGCCPDDSL

SEQ ID NO: 131 - protein, 17 aa, Artificial
  Synthetic
SGSGVFRCTGCCGDESL

SEQ ID NO: 132 - protein, 12 aa, Artificial
  Synthetic
SGSGNVFNEESL

SEQ ID NO: 133 - protein, 18 aa, Artificial
  Synthetic
SGSGNVFAAGAAANEESL

SEQ ID NO: 134 - protein, 19 aa, Artificial
  Synthetic
SGSGVNVFRCGGCCNEESL

SEQ ID NO: 135 - protein, 19 aa, Artificial
  Synthetic
SGSGFRCGGCCNEESLICM

SEQ ID NO: 136 - protein, 19 aa, Artificial
  Synthetic
SGSGTSYISKQLFEISVPL

SEQ ID NO: 137 - protein, 19 aa, Artificial
  Synthetic
SGSGISKQLFEISVPLTSV

SEQ ID NO: 138 - protein, 19 aa, Artificial
  Synthetic
SGSGQLFEISVPLTSVPEL

SEQ ID NO: 139 - protein, 19 aa, Artificial
  Synthetic
SGSGKVANHTGCKCLPTAP

SEQ ID NO: 140 - protein, 19 aa, Artificial
  Synthetic
SGSGNHTGCKCLPTAPRHP

SEQ ID NO: 141 - protein, 20 aa, Artificial
  Synthetic
SGSGASISARQDYKDDDDKT

SEQ ID NO: 142 - protein, 12 aa, Artificial
  Synthetic
SGSGDYKDDDDK

SEQ ID NO: 143 - protein, 31 aa, Artificial
  Synthetic
SGSGFFKPPCVNVFRCGGCCNEESLICMNTS

SEQ ID NO: 144 - protein, 5 aa, Artificial
  Synthetic
NEESL

SEQ ID NO: 145 - protein, 117 aa, Artificial
  Synthetic
DYKDDDDKFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYS

SEQ ID NO: 146 - DNA, 25 bp, Artificial
  Synthetic
ggtggctgttgccaagaagagagcc

SEQ ID NO: 147 - DNA, 25 bp, Artificial
  Synthetic
ggctctcttcttggcaacagccacc

SEQ ID NO: 148 - DNA, 25 bp, Artificial
  Synthetic
ctgttgcaatgatgagagccttatc

SEQ ID NO: 149 - DNA, 25 bp, Artificial
  Synthetic
gataaggctctcatcattgcaacag

SEQ ID NO: 150 - DNA, 25 bp, Artificial
  Synthetic
ctgttgcaatagcgagagccttatc

SEQ ID NO: 151 - DNA, 25 bp, Artificial
  Synthetic
gataaggctctcgctattgcaacag

SEQ ID NO: 152 - DNA, 22 bp, Artificial
  Synthetic
gcaatgaagacagccttatctg

SEQ ID NO: 153 - DNA, 22 bp, Artificial
  Synthetic
cagataaggctgtcttcattgc

SEQ ID NO: 154 - DNA, 21 bp, Artificial
  Synthetic
caatgaagagacccttatctg

SEQ ID NO: 155 - DNA, 21 bp, Artificial
  Synthetic
cagataagggtctcttcattg

SEQ ID NO: 156 - DNA, 22 bp, Artificial
  Synthetic
gcaatgaagagggccttatctg

SEQ ID NO: 157 - DNA, 22 bp, Artificial
  Synthetic
cagataaggccctcttcattgc

SEQ ID NO: 158 - DNA, 25 bp, Artificial
  Synthetic
caatgaagagagcgttatctgtatg

SEQ ID NO: 159 - DNA, 25 bp, Artificial
  Synthetic
catacagataacgctctcttcattg

SEQ ID NO: 160 - protein, 15 aa, Artificial
  Synthetic
CAAAANEESLAAAAA

SEQ ID NO: 161 - protein, 5 aa, Artificial
  Synthetic
NSEGL